(19) United States
(12) Patent Application Publication
(10) Pub. No.: US 2024/0407403 A1
(43) Pub. Date: Dec. 12, 2024

(54) A UV GERMICIDAL TREATMENT SYSTEM FOR OF OPAQUE LIQUIDS

(71) Applicant: LYRAS DK APS, Aalborg (DK)

(72) Inventors: Rasmus MORTENSEN, Aalborg (DK); Kristensen MATHIAS, Gistrup (DK)

(73) Assignee: LYRAS DK APS, Aalborg (DK)

(21) Appl. No.: 18/697,540

(22) PCT Filed: Sep. 28, 2022
(86) PCT No.: PCT/EP2022/076976

§ 371 Date: Apr. 1, 2024

(30) Foreign Application Priority Data
Oct. 1, 2021 (DK) PA202170485

(51) Int. Cl. A23L 3/28

(52) U.S. Cl. CPC A23L 3/28

(57) ABSTRACT

A system for a UV germicidal treatment, which enables a germicidal treatment of opaque liquids utilizing UV-C light, primarily in the wavelength between 180 nm to 300 nm. A surveillance system to include into a system capable of germicidal treatment of opaque liquids, such as highly opaque liquids sensitive to UV overexposure.

### TECHNICAL FIELD

The invention relates to a system for a UV germicidal treatment, which enables a germicidal treatment of opaque liquids utilizing UV-C light, primarily in the wavelength between 180 nm to 300 nm. The invention relates to a surveillance system to include into a system capable of germicidal treatment of opaque liquids, such as highly opaque liquids sensitive to UV overexposure.

### BACKGROUND ART

UV systems have previous been used for pasteurization of liquid food products. Examples of such instruments can be found in US 2002/096648, which discloses a reactor for irradiating ultraviolet light into a fluid reaction medium. An irradiation chamber is connected to an inlet and an outlet, which allows the reaction medium to flow through the reactor while being exposed to ultraviolet light.

Another example of such an UV-reactor instrument is US 2004/248076, which discloses an apparatus and process for sterilization of liquid media by means of UV irradiation and short-time heat treatment.

Further, such instruments are known in WO2019057257 disclosing a system capable of a germicidal treatment of highly opaque liquids, featuring a filter, which prevents wavelengths above the UV-C spectrum reaching the liquid being treated.

Further, monitoring systems comprising various sensors has previously been used as a measure to measure the efficiency of UV systems to provide an adequate removal of pathogens and other target microorganisms from the treated liquid. It is the target of international industrial standards for UV system designs, such as the Önorm M 5873-1, for liquids such as drinking water to document a fixed relationship between the reduction of target microorganisms and sensor values.

However, there is still a need within the field to provide a germicidal treatment for liquid products that are sensitive to high UV exposure, i.e. how to optimize the killing of bacteria and viruses (i.e. pasteurization or sterilization) while avoiding overexposure of the liquid. This problem is especially important when treating opaque liquids as all the liquid will not be exposed to the UV light as it is typically only possible to extend the UV intensity field throughout a small fraction of the volume of such liquid, which can easily lead to a highly uneven UV exposure and subsequently uneven dose with some volumes of the treated liquid being overexposed and other volumes insufficiently treated.

Further, there is also still a need within the field to use a UV germicidal treatment system, which is able to reduce the amount of energy used during treatment of opaque liquids.

Further, there is a need to simplify such germicidal treatment making it possible to adapt the equipment for an individual task while avoiding over or under exposure of the opaque liquid hereby providing a sufficient treatment to germicide the liquid (enough energy added) while avoiding over exposure.

### SUMMARY

The purpose of the present invention is to ensure adequate monitoring and control of parameters relevant to the effect of a UV system for the purpose of treating opaque liquids, especially liquids sensitive to UV overexposure.

It is provided that the acceptable limits for operating conditions for a product to be treated by the system has been defined such as, but not limited to, a maximum and a minimum flow, expected head loss from inlet to outlet of the flow system and optimum UV source output as defined by a UV sensor value that measures the output of said UV light source. It is considered that the successful treatment of a liquid relies mainly on a UV dose defined as a product of the accumulated time and intensity of UV exposure of the discrete volumes of the liquid passing through the system.

Another object of the present invention is to be able to ensure that the operating conditions are within limits for a multitude of product flow strings functionally arranged in parallel and to compare sensor values of equal strings to detect deviations such as sensor uncertainty, leaks or other flow interruptions such as blockages resulting from loss of coil shape stability, clogging, or fouling.

Another object of the present invention is to monitor and control the thermal conditions of the lamps in such a way, that constant output and optimum lamp lifetime can be achieved.

Yet another object of the present invention is to provide accurate and sufficient data throughout the different stages of operation of such a UV system, especially throughout the germicidal treatment of liquids, to be able to both control and document that all parameters relevant to maintain a sufficient performance of the system are within the limits required to achieve such performance.

Lastly, it is also an object of the present invention to be able to detect any faults or disturbances, mechanical and electrical, that occur in the UV system or its sensors that may influence the ability of the system to maintain proper performance such as leaks in the fluid system or drifting of sensor values due to sensor faults or ageing that would cause the key parameters of the system operation as provided by the sensors to be unreliable.

The present invention relates to a UV germicidal treatment instrument comprising a surveillance system for treatment of opaque liquids, such as for cold pasteurization of opaque liquid food products. Thus, disclosed in a first aspect of the present invention is a UV germicidal treatment system for treatment of opaque liquids, wherein the UV germicidal treatment system comprises: one or more spiral-shaped tubes extending from an inlet end to an outlet end creating a fluidic pathway; one or more means for controlling a flowrate of the opaque liquids through the fluidic pathway when the UV germicidal treatment system is in use; one or more UV light sources illuminating the one or more spiral-shaped tubes, wherein the one or more light sources emit light in a wavelength range between 180-300 nm; and a surveillance system configured for monitoring and controlling parameters of the UV germicidal treatment system for optimizing the germicidal treatment of the opaque liquids when the UV germicidal treatment system is in use; wherein the surveillance system comprises one or more UV light sensors configured to monitor an UV output related characteristics of the one or more UV light sources and provide an output accordingly, and wherein the one or more UV light sensors are positioned in the UV germicidal treatment system such that the one or more UV light sensors directly or indirectly measures an UV light intensity substantially proportional to the UV light intensity illuminating the one or more spiral-shaped tubes when the UV germicidal treatment system is in use; wherein the surveillance system further comprises at least one flow sensor, wherein the flow sensor is configured to monitor a flow related characteristics of the opaque liquid within the fluidic pathway when the UV germicidal treatment system is in use and provide an output accordingly, and wherein the flow sensor is positioned at or in the inlet end or at or in the outlet end of the fluidic pathway; and wherein the surveillance system further comprises a controller configured for: receiving a first input relating to the UV output related characteristics of the one or more UV light sources; receiving a second input relating to the flow related characteristics of the opaque liquid within the fluidic pathway; determining an UV treatment condition for the UV germicidal treatment system based on the received first and second input and the opaque liquids to be treated; and controlling the one or more UV light sources and/or the one or more means for controlling the flowrate based on the determined UV treatment condition.

By determining an UV treatment condition for the UV germicidal treatment system is meant that the current UV treatment condition of the system is determined by the controller, e.g. the current output of the lamp or the current flow of the liquid, and based on this, the controller is able to adjust the system such that the output/input is equal to instructions provided to the controller based on the liquid to be treated.

The means of controlling the flow rate, such as a pump or an adjustable pressure applied on the liquid at the inlet adjusted by the use of for example a P, PI, or PID regulation algorithm, may be directly integrated in the controls of the UV system itself or controlled by a different method based on a digital or analogue signal from the UV system. It is recognized that the proper treatment of a liquid varies depending not only on liquid type but also by factors such as the level of microbiological contamination, as can be determined by a sample taken from the liquid prior to treatment, variations in the viscosity, and increased levels of fouling of the system during the treatment of a batch. These and other factors may change the requirements for flow and UV exposure during the treatment, for example by gradually increasing the UV intensity during production or increasing the minimum required flow for a very opaque liquid to increase mixing or decrease the flow for a less opaque liquid to increase exposure time. In many cases, the flow through the UV system will not be directly controlled by the UV system, as the flow depends on other connected equipment such as a filling machine to bottle the product, that run at fixed flow patterns different from the requirements of the flow through the UV system. Such equipment may start or stop independently from the UV system. It is a solution to have a buffer tank between the UV system and the filling machine, so that a flow can be upheld through the UV system during short stops. However, following a stop, it may be desirable to increase flow through the UV system to reach a defined level in the tank, and if there is less stops than anticipated, and the buffer tank is filled, the flow through the UV system is reduced. In this case, it is desirable to allow fluctuations in flow through the UV system, and to monitor, that the flow fluctuations stay between a defined minimum and maximum known to support adequate treatment. If the flow falls outside defined levels, and alarm may be issued.

In a UV system used for drinking water, wastewater, or similar, the UV sensors are placed in such a way, that the UV light emitted from the lamp passes through the liquid before reaching the sensor. In this way, it is established with a high degree of certainty, that the minimum intensity in the liquid situated between the sensor and the lamp will be subject to an intensity that is at least similar to the value measured by the sensor corrected by the UV sensor uncertainty.

For a liquid product in which high UV exposure does not have any undesired effects, like water, it is sufficient to establish that a minimum UV intensity and a maximum volume flow have been observed throughout the treatment for it to be deemed successful.

For liquid products that are sensitive to high UV exposure or in order to save energy, it can be an advantage to dynamically adjust the UV output of the lamps to match the minimum requirements at varying flows or product qualities or as the UV lamp energy efficiency decreases during the ageing of the lamp.

One possible solution to this problem is presented in CN211019327U, in which a UV stable output system is described in which a feed back loop comprised of a UV lamp, a UV intensity sensor and a PLC controlling the lamp output can keep the value of the UV sensor constant.

However, such a system is not optimally designed to treat opaque liquids, at it is not possible to position the UV sensor in such a way, that the light received by the sensor has passed through the liquid, as the liquid layer absorbs UV light and thus prevents enough UV light to reach the sensor to achieve meaningful sensor values. Thus, a UV sensor to provide feedback for controlling the UV output must be placed in a position to monitor the light source UV output directly and not through a layer of liquid. This results in a further challenge, as the UV sensor value is no longer a measure of the minimum intensity in the liquid.

If one knows the flow pattern and geometry of the liquid volume in the coils of such a UV system, a UV dose could be indirectly derived from the measured flow and the sensor value by passing a sufficiently thin layer of the liquid between the light source and the sensor thin enough to allow a substantial fraction of the UV light to reach the sensor. However, the amount of UV light reaching the liquid through the walls of the coils depend not only on the transparency of the liquid to the desired UV wave lengths, but also on potential fouling of the internal surfaces of the coils, which could block some or all of the light from reaching the liquid.

Fouling is a phenomena that depends on specific liquid flow patterns, energy flows, and geometry, and so the fouling rate of the internal coil surface cannot be assumed to be similar to that of the boundary surfaces of the thin film flow passing between the light source and the sensor.

In a traditional UV system such as described in the Önorm M 5873-1, as disclosed in the background, it is assumed that at constant UV sensor value a lower flow will lead to a higher UV exposure time and subsequently a higher UV dose and a higher reduction of target microorganisms. It is assumed, that the effect of changes in turbulence and flow patterns inside the UV chamber that could lead to unfavorable changes in distribution of dose of the individual volumes of water passing through the system is at least counter balanced by the increase of average retention time and subsequently higher average UV dose. However, in a UV germicidal treatment system as described herein, this has been demonstrated not to be true. The reason for this is that in a typical application, due to the high UV absorbance properties of a typical liquid product to be treated, the UV light will only have effective penetration into a very small fraction of the liquid volume near the surface of the coil. The relative exchange rate of discrete volumes of product in- and out of this zone by means of turbulent energy is highly flow dependent and will typically be reduced at lower product flow speeds/rates thereby causing a loss in efficiency due to undesired distribution of doses experienced by the discrete volumes of liquid, causing more to be either overexposed or underexposed.

In a system as disclosed herein it is therefore insufficient to monitor a maximum flow and a minimum UV light intensity at any point in the system and to keep the UV output as measured by the UV sensor constant by regulating the power of the light source. It is also required to monitor minimum flow related to the properties of each individual liquid product to ensure efficient treatment. Further, it may be, depending on the liquid to be treated, be potentially advantageous to monitor the potential fouling of the internal surfaces of the coils as disclosed herein.

One of the advantages of using light radiation as a means for cold pasteurization is that it is a very energy efficient way for partial sterilization.

The fluidic pathway is designed to provide a high surface to volume ratio, increasing the exposure of light energy per unit volume with reduced self-shadowing effects from the opaque liquid being treated. In this manner it is possible to treat opaque liquids using light when the material, creating the fluidic pathway, is transparent to the radiation of light.

The opaque liquid food product flows through the one or more spiral-shaped tubes with a flow rate. In one or more embodiments, the flow rate measured in millilitres per minutes is between 200-20,000 ml/min, or between 500-15,000 ml/min, or between 800-12,500 ml/min, or between 900-10,000 ml/min.

By opaque liquid products is meant liquids in which the flux of UV light at 254 nm from a collimated beam of UV light is reduced by at least 95% by passing through 10 mm of the liquid, such as at least 98%.

Controlling the lamp output and the flow rate will ensure a uniform treatment of the opaque liquid to be treated. The UV light sensor values (the UV output as measured by the UV sensors) can be included in an instruction for the controller to follow along with a minimum pressure or flow rate. In other words, instructions for the controller can be defined by which UV sensor value that particular product should be treated with. Further, the flow sensor will ensure that a minimum flow rate is kept within the pathway, and that this minimum flow rate is sufficient to achieve sufficient turbulent mixing of the liquid to ensure proper exchange of discrete volume of liquid into the small fraction of the fluid pathway that is illuminated by UV light, increasing the exposure of light energy per unit volume with reduced self-shadowing effects from the opaque liquid being treated while simultaneously avoiding overexposure of UV light to the opaque liquid and prevent fouling inside the spiral shaped tubes.

Further, by the one or more means for controlling the flow rate being controlled based on information from the UV germicidal treatment system the system ensures that an optimal treatment for the opaque liquid is provided.

Disclosed herein in a second aspect of the present invention is a use of a UV germicidal treatment system for cold pasteurization of opaque liquid products, such as an opaque liquid food product, wherein the UV germicidal treatment system comprises: one or more spiral-shaped tubes extending from an inlet end to an outlet end creating a fluidic pathway; one or more means for controlling a flowrate of the opaque liquids through the fluidic pathway when the UV germicidal treatment system is in use; one or more UV light sources illuminating the one or more spiral-shaped tubes, wherein the one or more light sources emit light in a wavelength range between 180-300 nm; and a surveillance system configured for monitoring and controlling parameters of the UV germicidal treatment system for optimizing the germicidal treatment of the opaque liquids when the UV germicidal treatment system is in use; wherein the surveillance system comprises one or more UV light sensors configured to monitor an UV output related characteristics of the one or more UV light sources and provide an output accordingly, and wherein the one or more UV light sensors are positioned in the UV germicidal treatment system such that the one or more UV light sensors directly or indirectly measures an UV light intensity substantially proportional to the UV light intensity illuminating the one or more spiral-shaped tubes when the UV germicidal treatment system is in use; wherein the surveillance system further comprises at least one flow sensor, wherein the flow sensor is configured to monitor a flow related characteristics of the opaque liquid within the fluidic pathway when the UV germicidal treatment system is in use and provide an output accordingly, and wherein the flow sensor is positioned at or in the inlet end or at or in the outlet end of the fluidic pathway; and wherein the surveillance system further comprises a controller configured for: receiving a first input relating to the UV output related characteristics of the one or more UV light sources; receiving a second input relating to the flow related characteristics of the opaque liquid within the fluidic pathway; determining an UV treatment condition for the UV germicidal treatment system based on the received first and second input and the opaque liquids to be treated; and controlling the one or more UV light sources and/or the one or more means for controlling the flowrate based on the determined UV treatment condition.

Cold pasteurization may be partial sterilization of a substance and especially a liquid in a process where heat is evaded as the main eradication of objectionable organisms without major chemical alteration of the substance. With evaded is not meant excluded but reduced.

In one or more embodiments, a biological contaminant is inactivated or reduced by an order of at least 2-Log10. A biological contaminant may be e.g., bacteria, spores, mold, or virus.

In one or more embodiments, a biological contaminant is inactivated or reduced by an order of at least 3-Log10. In another embodiment, a biological contaminant is inactivated or reduced by an order of at least 4-Log10. In another embodiment, a biological contaminant is inactivated or reduced by an order of at least 5-Log10. In yet another embodiment, a biological contaminant is inactivated or reduced by an order of at least 6-Log10.

Disclosed herein in a third aspect of the present invention is a use of a UV germicidal treatment system for killing microorganisms, such as bacteria, mold, spores, or virus, in opaque liquid products, such as an opaque liquid food product, wherein the UV germicidal treatment system comprises: one or more spiral-shaped tubes extending from an inlet end to an outlet end creating a fluidic pathway; one or more means for controlling a flowrate of the opaque liquids through the fluidic pathway when the UV germicidal treatment system is in use; one or more UV light sources illuminating the one or more spiral-shaped tubes, wherein the one or more light sources emit light in a wavelength range between 180-300 nm; and a surveillance system configured for monitoring and controlling parameters of the UV germicidal treatment system for optimizing the germicidal treatment of the opaque liquids when the UV germicidal treatment system is in use; wherein the surveillance system comprises one or more UV light sensors configured to monitor an UV output related characteristics of the one or more UV light sources and provide an output accordingly, and wherein the one or more UV light sensors are positioned in the UV germicidal treatment system such that the one or more UV light sensors directly or indirectly measures an UV light intensity substantially proportional to the UV light intensity illuminating the one or more spiral-shaped tubes when the UV germicidal treatment system is in use; wherein the surveillance system further comprises at least one flow sensor, wherein the flow sensor is configured to monitor a flow related characteristics of the opaque liquid within the fluidic pathway when the UV germicidal treatment system is in use and provide an output accordingly, and wherein the flow sensor is positioned at or in the inlet end or at or in the outlet end of the fluidic pathway; and wherein the surveillance system further comprises a controller configured for: receiving a first input relating to the UV output related characteristics of the one or more UV light sources; receiving a second input relating to the flow related characteristics of the opaque liquid within the fluidic pathway; determining an UV treatment condition for the UV germicidal treatment system based on the received first and second input and the opaque liquids to be treated; and controlling the one or more UV light sources and/or the one or more means for controlling the flowrate based on the determined UV treatment condition.

With killing is meant reducing the amount of active, viable, and/or living microorganisms. Microorganisms found in liquid food products may be present due to contamination during the processing of said liquid food product. Common bacteria contamination of e.g. dairy products may be e.g., Lactobacillus casei, Escherichia coli, Listeria monocytogenes, Salmonella spp., Mycobacterium avium subspecies paratuberculosis (MAP), Staphylococcus aureus, or Streptococcus spp.

Disclosed herein in a fourth aspect is a surveillance system configured for monitoring and controlling parameters of a UV germicidal treatment system for optimizing the germicidal treatment of the opaque liquids when the UV germicidal treatment system is in use; wherein the surveillance system comprises one or more UV light sensors configured to monitor an UV output related characteristics of one or more UV light sources and provide an output accordingly; wherein the surveillance system further comprises at least one flow sensor, wherein the flow sensor is configured to monitor a flow related characteristics of an opaque liquid within a fluidic pathway when the UV germicidal treatment system is in use and provide and output accordingly; and wherein the surveillance system further comprises a controller configured for: receiving a first input relating to the UV output related characteristics of the one or more UV light sources; receiving a second input relating to the flow related characteristics of the opaque liquid within the fluidic pathway; determining an UV treatment condition for the UV germicidal treatment system based on the received first and second input and the opaque liquids to be treated; and controlling the one or more UV light sources and/or the one or more means for controlling the flowrate based on the determined UV treatment condition.

Disclosed herein in a fifth aspect is a use of the surveillance system according to aspect 4 for optimizing germicidal treatment of opaque liquids treated in an UV germicidal treatment system.

Disclosed herein in a sixth aspect is a method for optimizing germicidal treatment of opaque liquids in an UV germicidal treatment system, wherein the method comprises the following steps:
providing the opaque liquid through a fluidic pathway in the UV germicidal treatment system;
controlling a flow rate of the opaque liquids through the fluidic pathway via one or more means for controlling the flow rate;
illuminating the fluidic pathway by emitting light in a wavelength range between 180-300 nm from one or more UV light sources;
   monitor and control parameters of the UV germicidal treatment system via a surveillance system comprising one or more UV light sensors, at least one flow sensor, and a controller, wherein the UV light sensors monitor an UV output related characteristics of the one or more UV light sources and provide an output accordingly and the at least one flow sensor monitor a flow related characteristics of the opaque liquid within the fluidic pathway and provide an output accordingly, wherein the monitor and control comprises the following steps;
provide a first input relating to the UV output related characteristics of the one or more UV light sources to the controller;
provide a second input relating to the flow related characteristics of the opaque liquid within the fluidic pathway to the controller;
determine an UV treatment condition for the UV germicidal treatment system based on the received first and second input and the opaque liquids to be treated; and
control the one or more UV light sources and/or the one or more means for controlling the flowrate based on the determined UV treatment condition.

### DETAILED DESCRIPTION OF THE INVENTION

The present disclosure will become apparent from the detailed description given below. The detailed description disclose preferred embodiments of the disclosure by way of illustration only. Those skilled in the art understand from guidance in the detailed description that changes and modifications may be made within the scope of the disclosure.

The invention relates to a UV germicidal treatment system comprising a surveillance system, which enables a germicidal treatment of opaque liquids utilizing UV-C light, ranging from 180 nm to 300 nm.

In describing the aspects of the invention, specific terminology will be used for the sake of clarity. However, the invention is not intended to be limited to the specific terms so selected, and it is understood that each specific term includes all technical equivalents, which operate in a similar manner to accomplish a similar purpose.

Disclosed herein is a UV germicidal treatment system comprising a surveillance system for treatment of opaque liquids, such as for cold pasteurization of opaque liquid food products. The system further comprises one or more spiral-shaped tubes, one or more means for controlling a flowrate, one or more UV light sources. The surveillance system is configured for monitoring and controlling parameters of the UV germicidal treatment system for optimizing the germicidal treatment of the opaque liquids when the UV germicidal treatment system is in use by using one or more UV light sensors, at least one flow sensor, and a controller. The controller is configured for receiving a first input from the one or more UV light sensors and the at least one flow sensor and based on these two inputs and the opaque liquids to be treated determine an optimal treatment condition for said liquid and hereafter control the light sources and/or the one or more means for controlling the flowrate to ensure that the optimal treatment conditions are obtained.

The invention further relates to the use of the UV germicidal treatment system for cold pasteurization of opaque liquid products, and for killing microorganisms, such as bacteria, mold, spores, or virus, in opaque liquid products.

The invention also relates to a surveillance system configured for monitoring and controlling parameters of a UV germicidal treatment system for optimizing the germicidal treatment of the opaque liquids when the UV germicidal treatment system is in use, the use of said surveillance system for optimizing germicidal treatment of opaque liquids treated in an UV germicidal treatment system, and a method for optimizing germicidal treatment of opaque liquids in an UV germicidal treatment system.

Pasteurization is not only limited to partial sterilization of a substance and especially a liquid at a temperature and for a time period of exposure that destroys objectionable organisms without major chemical alteration of the substance, but also covers cold pasteurization which is partial sterilization of a substance and especially a liquid in a process where heat is evaded as the main eradication of objectionable organisms without major chemical alteration of the substance. With evaded is not meant excluded but reduced. The present invention discloses that one of the advantages of using light radiation as a means for cold pasteurization is that it is a very energy efficient way for partial sterilization.

The fluidic pathway is designed to provide a high surface to volume ratio compared to traditional systems used for less opaque liquids, which allows the UV light to penetrate the liquid deeper, increasing the exposure of light energy per unit volume with reduced self-shadowing effects from the opaque liquid being treated. In this manner it is possible to treat opaque liquids using light when the material, creating the fluidic pathway is transparent to the radiation of light.

The one or more spiral-shaped tubes extending from an inlet end to an outlet end creating a fluidic pathway utilizes the flow regime occurring when the media is traveling in the fluidic pathway. The flow regime in the fluidic pathway may consists of one or several eddies, which creates a secondary flow perpendicular to the primary flow utilizing the centrifugal force (e.g. Dean vortex flow) to enhance the surface of the liquid being exposed to UV-light emitted by the light sources.

The fluid movement through the fluidic pathway may be at a flowrate that facilitates turbulent flow. However, the fluid movement through the fluidic pathway may alternatively be performed at a flowrate that facilitates a double vortexual pattern consistent with a Dean vortex flow. This provides an axial flow in the fluidic pathway, providing a high surface to volume ratio providing a high exchange rate of the liquid at the relatively small illuminated area near the wall of the fluidic pathway. This may increase the exposure of light energy per unit volume/surface area with reduced self-shadowing effects from the opaque liquid being treated.

In one or more embodiments, the controller is configured for controlling the one or more UV light sources to keep the UV output of the one or more UV light sources on a predefined value.

In one or more embodiments, the one or more means for controlling the flowrate is selected from one or more pumps, one or more valves, one or more pressurized tank systems, or a combination hereof.

In one or more embodiments, at least one flow sensor is selected from a flowrate sensor, a pressure sensor, or combinations hereof.

As the fluid passes through the fluidic pathway of the coil, energy is absorbed from the friction between the fluid and the pathway wall along with internal friction in the liquid and kinetic energy stored in the turbulence of the flow. This results in a hydrodynamic resistance that is overcome by establishing a pressure difference between the inlet and outlet of the pathway to drive the flow through the coiled tube. It is assumed, if the physical properties of the liquid and the liquid pathway are constant there is a fixed relationship between the flow speed of the liquid through the pathway and the pressure difference between the inlet and the outlet of the pathway that is driving the flow. This relationship can be known and registered by the controller if sensor values are obtained from a flow meter and from pressure meters placed at the inlet and at the outlet. The pressure required to overcome the resistance required to drive the flow can be found by subtracting the recorded pressure at the outlet from the recorded pressure at the inlet.

As the relationship between the pressure drop (the “head loss”), of the system and the flow is known in a constant state, deviations from this relationship will represent a change in either the physical properties of the liquid, such as a change in viscosity or a change in the physical properties of the fluidic pathway such as fouling on the tube wall, resulting in either reduced diameter of the pathway, added friction due to increase in surface roughness, or both; or a change in volume of the pathway due to expansion from excess levels of combined heat and pressure;

or leaks. As the physical properties of the liquid is often known, the flow/pressure relationship can be used to monitor the state of the fluidic pathway and provide a method to control the lamp output, for example by increasing the lamp output at increased fouling rates, or issue alarms.

The pressure loss from inlet to outlet converts to friction and turbulent energy internally in the liquid and friction between the liquid and the fluidic pathway, and these effects are ensuring mixing of the liquid and subsequently the exchange rate in and out of the UV intensity zone of the pathway. Thus, as there is normally a fixed relationship between the pressure loss and the flow, pressure meters at the inlet and outlet can be used as an alternative to flow meters as an indicator for the efficiency of the treatment. Given a specific liquid with known physical characteristics, a required microbiological effect of the treatment and the geometry of the fluidic pathway, a minimum head loss or a minimum flow in combination with a related UV intensity can be defined that is required to perform an adequate treatment and it is further possible to improve the system performance by adjusting the intensity of the UV source based on both the values of the flow or the values of the pressure loss or the ratio between head loss and flow or combinations thereof.

In one or more embodiments, the controller is configured with a predefined minimum value and a predefined maximum value for determining the UV treatment condition, and wherein the controller is configured to determine the UV treatment condition based on the received first and second input, such that the UV treatment condition is within the predefined minimum and maximum value.

In many cases, the properties of the liquid to be treated is not constant, often due to variations of viscosity. It is also often the case that an increase in flow or pressure loss from a defined minimum required to achieve efficient treatment results in increased efficiency due to better mixing of the liquid and higher exchange rate to and from the zone of UV intensity. As the higher efficiency compensates for the lower average exposure time, caused by lower retention time in the fluidic pathway, the effect of the smaller UV dose on the target organisms may be similar to or better than that of the defined minimum flow or pressure drop. It is also the case, that there is a maximum flow or pressure drop defined by the mechanical properties of the fluidic pathway or a turning point, where the added efficiency no longer adequately compensates the lower retention time. For this reason, it is desirable to monitor that the flow or pressure loss stays within a minimum and a maximum value applicable to the individual treatment parameters. This is also an advantage, as it allows the flow to vary within the defined boundaries so that it can be adjusted to meet the flow requirements of up- or downstream equipment.

In one or more embodiments, the controller is configured to control both the one or more UV light sources and the one or more means for controlling the flowrate, and wherein the controller is further configured to control the one or more UV light sources and/or the one or more means for controlling the flowrate based on the determined UV treatment condition.

In one or more embodiments, the UV germicidal treatment system is for avoiding overexposure of opaque liquids to be treated.

It is critical for some liquids like milk, soy products, beer, and many other beverages, to avoid overexposure, as the energy of the UV photons can cause compounds like riboflavin to be oxidized hereby producing by-products with undesired taste and smell that will potentially render the product unsuitable for consumption.

In one or more embodiments, the UV germicidal treatment system is for reducing the amount of energy used by the UV germicidal treatment during treatment of opaque liquids.

It is also considered to be importance to minimize the energy consumption of the system due to multiple factors such as environmental impact, return of investment time for the customer, and undesired excess heat discharged from the cooling system of the UV system to the room where the system is installed or absorbed by the treated liquid.

In one or more embodiments, the UV germicidal treatment system further comprises one or more valves configured to change the fluidic pathway within the one or more spiral-shaped tubes and one or more additional internal UV light sensors positioned inside the fluidic pathway of the one or more spiral-shaped tubes, wherein the additional internal UV light sensors are configured to monitor an UV output related to opaque characteristics of the liquid within the fluidic pathway during use and provide an output accordingly, and wherein the controller is further configured for: receiving a third input relating to the UV output related to opaque characteristics of the liquid, determining if the opaque characteristics of the liquid changes, and controlling the one or more valves such as changing the fluidic pathway of the one or more spiral-shaped tubes within the UV germicidal treatment system if the opaque characteristics of the liquid changes.

In one or more embodiments, the UV germicidal treatment system further comprises one or more additional internal UV light sensors positioned such that the one or more additional internal UV light sensors are exposed only to UV radiation that has passed through the one or more spiral-shaped tubes, wherein the additional internal UV light sensors are configured to monitor an UV output related to opaque characteristics of the liquid within the fluidic pathway during use and provide an output accordingly, and wherein the controller is further configured for: receiving a third input relating to the UV output related to opaque characteristics of the liquid, determining if the opaque characteristics of the liquid changes, and controlling the one or more valves such as changing the fluidic pathway of the one or more spiral-shaped tubes within the UV germicidal treatment system if the opaque characteristics of the liquid changes.

In one embodiment such a position is on a point on the axis of the spiral shaped liquid pathway tube at or near the center of the axis of the spiral. To ensure that all light that is registered by the sensor has passed through both the tube and the fluidic pathway, the spiral must be compressed in the direction of the axis to ensure no gaps form between the individual revelations of the tube. This is achieved in one instance by orienting the spiral shaped tube such that the axis is vertical and gravity pulls the revelations together to close gaps or to make sure any gaps that forms between revelations due to variations or imperfections on the coiled tube, will remain constant and thus cause a constant bias to the measured sensor value.

The spiral shaped tube can be positioned around a solid pipe, for example made from steel, that has a shared center axis with the spiral shaped tube and that has an outer diameter similar to or slightly smaller than the diameter of the spiral through the tube centers minus the tube external diameter. Such a tube will stabilize the spiral shaped tube and a UV sensor may be fixed inside the tube at a position, where the tube is perforated to allow UV light that has passed through the tube and liquid to reach the sensor.

The UV light will reach the one or more additional internal UV light sensors through the spiral-shaped tubes, hereby UV light will be blocked, at least partly, if the tubes are filled with an opaque liquid, i.e. an UV absorbent product. These values may range from about 500 W/m2, such as about 300 W/m2, if water is provided to the tubes (which is substantially transparent to UV light from 180 to 300 nm) to about 8 W/m2 if e.g. milk is provided in the tubes (which a highly opaque liquid, hereby almost completely absorbing all UV light). The advantage of recognizing a change as described is that the system will recognize when a product, i.e. an opaque liquid, is present in the tubes, such that if a cleaning step with water is provided product waste is minimized, as the system can inform a valve that there is now product in the system, hereby switching from waste to product collection. In addition, it allows the controller to only monitor the treatment (UV values, pressure, etc.) only when the system can see that the product is being processed, hereby also avoiding providing an alarm to the user if it is water in the tubes cleaning the system.

In one or more embodiments, the controller is further configured for determining a first ratio between the UV output related to opaque characteristics of the liquid within the fluidic pathway and the UV output related characteristics of the one or more UV light sources at an initial state of the UV germicidal treatment system in which the spiral-shaped tubes are clean and in which a standard liquid transparent to UV, such as water, is provided within the fluidic pathway, and wherein this first ratio is defined as a clean state ratio of the UV germicidal treatment system.

Depending on the opaque liquid to be treated, the spiral-shaped tubes may over time be contaminated with fouling on the inside of said tubes where the fluidic pathway is provided. Adequate UV treatment can only be detected if there has been sufficient UV intensity throughout the treatment and if the tubes have been sufficiently clean during the treatment. The post-treatment ratio when the tubes are again filled with clean water between the UV intensity measured directly from the lamps and the UV intensity measured through the coils of the internal sensor will be an indication of the degree of fouling that has occurred during production (provided the coatings are not completely water-soluble. The time and flow rate of water from the end of production to the measured fouling must be the same to compare productions 1:1.

In one or more embodiments, the UV germicidal treatment system is further configured for a predefined period of UV germicidal treatment of opaque liquids followed by a predefined period of flushing the UV germicidal treatment system with standard liquid transparent to UV, such as water, wherein the controller is further configured for determining a second ratio between the UV output related to opaque characteristics of the liquid within the fluidic pathway and the UV output related characteristics of the one or more UV light sources after the predefined period of flushing when the standard liquid transparent to UV, such as water, is provided within the fluidic pathway, and comparing the ratio with the clean state ratio for determining and quantify a rate of fouling or determine a return to a clean state.

In one or more embodiments, the controller is configured for providing an alarm to a user if the second input relating to the flow related characteristics of the opaque liquid within the fluidic pathway is below a predefined minimum value.

This ensures that a minimum flow rate is kept to provide the necessary turbulence to expose the liquid to the UV light while simultaneously avoid overexposure of said liquid to the UV light and to prevent fouling inside the tubes.

In one or more embodiments, the at least one flow sensor is at least two flow sensors, wherein at least one flow sensor is selected from a pressure sensor and at least one other flow sensor is selected from a flowrate sensor.

In one or more embodiments, the at least one flow sensor is at least three flow sensors, wherein at least two flow sensors are selected from a pressure sensor and at least one other flow sensor is selected from a flowrate sensor.

In one or more embodiments, the at least one flow sensor is a pressure sensor, wherein the flow sensor is positioned at or in the inlet end of the fluidic pathway, and wherein the controller is configured for providing an alarm to a user if the second input relating to the flow related characteristics of the opaque liquid within the fluidic pathway is below a predefined minimum value.

Pressure can be measured, as there is a correlation between pressure and flow rate. The UV germicidal treatment system will experience a significant loss of pressure from inlet to outlet. This loss in pressure is correlated to the friction and turbulence created in the spiral-shaped tubes fluidic pathway. The more opaque the liquid is the higher expected pressure needs to be applied to uphold an effective treatment.

In one or more embodiments, the at least one flow sensor is at least two flow sensors, wherein at least one flow sensor is positioned at or in the inlet end of the fluidic pathway and at least one other flow sensor is positioned at or in the outlet end of the fluidic pathway.

In one or more embodiments, at least one flow sensor is positioned at or in the outlet end of the fluidic pathway.

If the flow sensor is positioned at or in the outlet end of the fluidic pathway the sensor will also be able to tell the user if a leakage within the tubes is occurring. It is therefore also suboptimal to only have a flow sensor at the inlet end of the fluidic pathway, as leakages in some instances will not be caught by the system.

In one or more embodiments, the controller is configured for receiving an input from the at least two flow sensors, determining a ratio between the input from the at least two flow sensors, and providing an alarm to a user if the ratio between the input from the at least two flow sensors is above a predefined maximum ratio or below a predefined minimum ratio.

One of the advantages of such a solution is that this allows the user to detect changes in flow caused by clogging, fouling, leaks, or changes in the viscosity of the opaque liquid, e.g. if milk is processed and cream is at the top of the tank, or if blood plasma stands and coagulates over time in the tank that supplies the UV system with the liquid, or if there are temperature changes in liquids where the viscosity is very temperature dependent.

In one or more embodiments, the at least one flow sensor comprises at least two flow sensors both positioned at or in the inlet end or at or in the outlet end of the fluidic pathway and wherein the controller is configured to continuously compare deviations in flow related output of the two flow sensors.

If the viscosity is the cause of a change in flow for a particular pressure, the effect will be the same for all the tubes in the system. Leaks and clogs could be asymmetrical. Fouling could theoretically be both symmetrical and asymmetrical, but would most likely be symmetrical.

In one or more embodiments, the controller is configured for increasing or decreasing the light emitted by the one or more UV light sources illuminating the one or more spiral-shaped tubes if the controller determines drop or increase in the second input relating to the flow related characteristics of the opaque liquid within the fluidic pathway.

In one or more embodiments, the UV germicidal treatment system further comprises one or more filters positioned between the one or more light sources and the one or more spiral-shaped tubes, wherein the one or more filters prevent light above a wavelength of 300 nm from reaching the one or more spiral-shaped tubes.

By preventing light above a wavelength of 300 nm from reaching the one or more spiral-shaped tubes is meant that light above 300 nm, but below 500 nm (300-500 nm), is attenuated by a substantial amount, e.g. at least a factor of 100, or a factor of 1,000 or more.

In one or more embodiments, the one or more filters prevent light above a wavelength of 280 nm from reaching the one or more spiral-shaped tubes.

One of the advantages using one or more filters is that photo oxidation from higher wavelengths may be avoided. E.g. avoiding photo oxidation of riboflavin (around a wavelength of 446 nm) is preferred, but also avoiding photo oxidation of other components in the liquid food product, which enhances a bitter and bad flavour/taste in said product, is preferred. Additionally, the filters may avoid hot air from contacting the one or more spiral-shaped coils, hereby avoiding heating of the liquid product. Even further, the filters may also serve as parts forming a channel for air to flow through, hereby assisting in cooling of the one or more light sources to obtain optimal processing temperature.

In one or more embodiments, the one or more filters are selected from band-pass filters, notch filters, or a combination of both.

A band-pass filter is a device that passes frequencies within a certain range and rejects/attenuates frequencies outside that range.

A notch filter is a band-stop filter with a narrow stopband. In signal processing, a band-stop filter or band-rejection filter is a filter that passes most frequencies unaltered, but rejects/attenuates those in a specific range to very low levels. It is the opposite of a band-pass filter.

In one or more embodiments, the system further comprises an adaptive cooling system comprising one or more blowing units for driving an airflow through the UV germicidal treatment system.

To ensure optimum operation of the UV light source cooling has to be applied to remove excess heat from the light source, as available light sources are not 100% efficient in converting electrical energy to UV output and most of the energy loss is converted to heat. Efficiency of light sources such as low pressure amalgam lamps are highly dependent on maintaining thermal balance and requires well designed cooling systems such as described herein. If amalgam lamp cooling is insufficient, the UV output drops significantly and if overcooled both lamp lifetime and UV output is reduced.

In a system with a simple feedback loop such as described in CN211019327U the lamps may be operating at less than full power. If in this state cooling becomes insufficient due to external temperatures rising, cooling air flow reduction due to clogged filters or other factors, the UV output may be momentarily reduced. The feedback loop will respond by increasing lamp power to increase output, but this in turn can cause further over heating and result in a reduced output. Such a cascade pose a problem, as the UV system may suffer a big loss in output and fail to adequately perform its treatment. Other light sources, such as UV emitting diodes, are less sensitive but require cooling to stay below a maximum temperature to prevent deterioration of its building materials.

In one or more embodiments, the one or more blowing units are configured for driving the airflow through the UV germicidal treatment system by creating a negative pressure inside the UV germicidal treatment system.

In one or more embodiments, the one or more blowing units are configured for driving the airflow through the UV germicidal treatment system by creating a positive pressure inside the UV germicidal treatment system.

In one or more embodiments, the adaptive cooling system further comprises one or more temperature sensors.

In one or more embodiments, the one or more temperature sensors are positioned at or in an air outlet or at or in an air outlet of the UV germicidal treatment system.

In one or more embodiments, the controller is configured for controlling the adaptive cooling system based on effect, such as Watt used, of the one or more UV light sources illuminating the one or more spiral-shaped tubes.

Virtually all power used by the system comes from the lamps (UV light source), and therefore it can give an advantage to adjust the fan speed/cooling power according to the power consumption of the lamps.

In one or more embodiments, the controller is configured for controlling the adaptive cooling system based on a received input from the one or more temperature sensors.

In one or more embodiments, the one or more temperature sensors are at least two temperature sensors positioned at different positions within the UV germicidal treatment system, and wherein the controller is configured for controlling the adaptive cooling system based on a difference between a received input from one of the at least two temperature sensors and a received input from another of the at least two temperature sensors.

One of the advantages of such a system is that by measuring a difference the fans will automatically work harder to maintain this temperature difference if it rises over time and there will be a positive correlation between the temperature difference and the temperature of the lamps. If the effect of the lamps is reduced because they are dimmed, the fan speed goes down as it should to maintain the temperature difference, at less heat is produced by the lamp, hence less cooling may be needed.

In one or more embodiments, the controller is configured for controlling the adaptive cooling system based on a received input from one or more temperature sensors positioned at or in an air outlet of the UV germicidal treatment system.

The advantage here is that the temperature of the surroundings is easily compensated for. As constant power is emitted from the lamps, the fan speed will increase if the blow-in temperature increases, as in this situation there is not such a large inlet/outlet temperature difference to utilize. A further advantage of this solution is that only one sensor is required in the cooling system.

Another advantage of controlling the cooling air flow based on a temperature sensor value from a sensor positioned in the outlet air stream is, that if for example the fans are regulated to maintain a constant outlet temperature, the warm-up time before the lamps reach their operating temperature and output is reduced, as the fans will stay at low or no power and only provide the full and appropriate cooling power, as the outlet air reaches the set temperature.

An additional advantage is that by monitoring the exhaust temperature, in case fans are pulling the hot air from the system and are exposed to the exhaust air, it can be ensured, that the maximum working temperature of the fans or other exposed electronic components are not exceeded.

In one or more embodiments, the controller is configured for controlling the adaptive cooling system based on a received input from one or more temperature sensors positioned at or in an air inlet of the UV germicidal treatment system.

If the power consumption of the system is known, this will allow the system to adjust the fan power to compensate for variations of the temperature of the inlet air. Another advantage of monitoring the inlet temperature is that an alarm may be issued if the inlet temperature falls below freezing temperature, which could pose a risk of harmful ice formation inside the UV system.

In one or more embodiments, the controller is configured for providing an alarm to a user if the controller receives an input from that the one or more temperature sensors above a predefined value.

In one or more embodiments, the controller is configured for providing an alarm to a user if the controller receives an input from that the one or more temperature sensors above a predefined value and receives a maximum capacity input from the one or more blowing units.

In one or more embodiments, the system further comprises an adaptive cooling system comprising one or more blowing units for driving an airflow through the UV germicidal treatment system; wherein the one or more blowing units are configured for driving the airflow through the UV germicidal treatment system by creating a negative or positive pressure inside the UV germicidal treatment system; wherein the adaptive cooling system further comprises one or more temperature sensors; wherein the controller is configured for controlling the adaptive cooling system based on effect, such as Watt used, of the one or more UV light sources illuminating the one or more spiral-shaped tubes or wherein the controller is configured for controlling the adaptive cooling system based on a received input from the one or more temperature sensors.

The system may further comprise a cassette system comprising different elements of the system. A cassette system may make it easier to change the light sources during service. As one cassette may be replaced without having to change anything else in the system, hence in one or more embodiments, the UV germicidal treatment system further comprises a first cassette mounting frame and at least two cassettes extending from a first end to a second end; wherein the cassette mounting frame comprises cassette receiving openings into which each of the cassettes are removable mounted; wherein each cassette comprises the one or more light sources illuminating the one or more spiral-shaped tubes; and wherein one or more of the one or more spiral-shaped tubes are positioned between two of at least two cassettes.

In a UV germicidal treatment system, it is preferred that as large a portion as possible of the UV light reaches the liquid. However, it is also preferable to minimize the visible light and heat radiation and heat transfer via convection to the liquid. By adding a filter, e.g. a band-pass filter, to exclude the unwanted wavelengths and by encapsulating the light sources into a cassette system both the above may be ensured. Further, if the system comprises a cassette system, the cassettes may comprise the one or more of the one or more filters; hence, in one or more embodiments, each cassette also comprises one or more of the one or more filters, such that the one or more filters are positioned between the one or more light sources and the one or more spiral-shaped tubes.

In one or more embodiments, the one or more of the spiral-shaped tubes are grouped in sets of at least two, such as sets of at least three, positioned in a configuration alternating between a set of one or more of the spiral-shaped tubes and a cassette.

In one or more embodiments, the UV germicidal treatment system further comprises a first ventilation chamber positioned at the first end of the one or more cassettes.

In one or more embodiments, the UV germicidal treatment system further comprises a second ventilation chamber positioned at the second end of the one or more cassettes.

In one or more embodiments, the ventilation chamber pulls air out of the cassette or at the ventilation chamber air flows into the cassette.

By drawing air into or out of the cassettes it removes the heat the light source produces. In addition, it is very important to get the most energy and lifespan out of the light sources. This means that they must be cooled evenly and uniformly to their optimum operating temperature. By having ventilation chambers in one or both ends of the cassettes a uniform and optimum operation temperature may be obtained.

In one or more embodiments, the ventilation chamber pulls air out of the cassette at both ends.

The cooling system of the cassettes may work by sucking/pulling air out of both ends. This creates a slightly reduced pressure inside the cassettes.

In one or more embodiments, at the ventilation chamber air flows into the cassette at both ends.

In one or more embodiments, the ventilation chamber pulls air out of the cassette at one end and air flows into the cassette at the other end.

In one or more embodiments, each of the cassettes further comprises air intake openings for allowing air to flow into the cassette.

In one or more embodiments, each of the cassettes further comprises a cassette frame with openings, wherein a first set of openings are covered by glass, such as quartz glass, through which light from the light sources can illuminate the one or more of the spiral-shaped tubes.

In one or more embodiments, each of the cassettes further comprises a cassette frame with openings, wherein a second set of openings are adapted for facilitating internal air movement inside the cassette.

The cassettes further comprise small openings in the cassette frames. These openings are designed to be small enough to maintain the negative pressure in the cassette, and they are positioned so that the air that enters cools the lamps uniformly. The openings may e.g. be sized to make the air entering the cassettes flow with a speed of approximately 2 m/s. This means that the air velocity ensures a turbulent stirring of the air in the cassette, which in turn ensures uniform cooling. It further ensures that if the vacuum is uniform within the cassette, air will enter through all the openings. If the openings were too large, air would only enter through the openings closest to where the air is sucked out.

The airs path to the openings may be designed so that UV light does not escape through the intake. This ensures that no or very little UV radiation reaches the surroundings, and that the one or more spiral-shaped tubes is not exposed to unfiltered light.

In one or more embodiments, the cassette comprises a plurality of openings, wherein an air flow is generated through the plurality of openings when a pressure difference is applied between an internal and external surface of the cassette, and wherein flow of air driven by said pressure difference through the plurality of openings provide a uniform cooling along the entire length of the one or more light sources in order to reach maximum UV output and ensure optimum life time of the one or more light sources.

The plurality of openings in the cassette can be used for cooling of the one or more light sources. The openings can be designed to ensure that if a small pressure difference between the cassette and the surrounding environment is applied it will generate a uniform flow of air through the entire cassette, hereby obtaining an optimal cooling of the one or more light sources. The external and internal surface of the cassette is the outside and inside surface of the cassette, respectively.

In one or more embodiments, the UV germicidal treatment system further comprises a first cassette mounting frame and at least two cassettes extending from a first end to a second end; wherein the cassette mounting frame comprises cassette receiving openings into which each of the cassettes are removable mounted; wherein each cassette comprises the one or more light sources illuminating the one or more spiral-shaped tubes; and wherein one or more of the one or more spiral-shaped tubes are positioned between two of at least two cassettes; wherein the UV germicidal treatment system further comprises a first ventilation chamber positioned at the first end of the one or more cassettes; wherein the UV germicidal treatment system further comprises a second ventilation chamber positioned at the second end of the one or more cassettes; wherein the first and/or second ventilation chamber pulls air out of the cassette or at the first and/or second ventilation chamber air flows into the cassette; wherein the cassette comprises a plurality of openings, wherein an air flow is generated through the plurality of openings when a pressure difference is applied between an internal and external surface of the cassette, and wherein flow of air driven by said pressure difference through the plurality of openings provide a uniform cooling along the entire length of the one or more light sources in order to reach maximum UV output and ensure optimum life time of the one or more light sources.

In one or more embodiments, a space between two cassettes of the UV germicidal treatment system or a space between a cassette and one or more of the spiral-shaped tubes functions as a ventilation shaft used for cooling of the UV germicidal treatment system, especially cooling the cassettes comprising the one or more light sources.

In one or more embodiments, the one or more light sources operate at a lamp temperature between 0° C. and 120° C., such as between 20° C. and 100° C., such as between 40° C. and 100° C., such as between 60° C. and 100° C., or such as between 80° C. and 100° C.

In one or more embodiments, the one or more filters are selected from band-pass filters, notch filters, or a combination of both.

In one or more embodiments, the UV germicidal treatment system further comprises a reactor housing.

The reactor housing is modularly designed, and hence does not have a minimum or maximum length. The size of the reactor housing may depend on the size of the cassettes, the one or more spiral-shaped tubes, and other features added to the system. A reactor housing may be desirable as it will contain the light inside the reactor and reflect the light back towards the one or more spiral-shaped tubes.

In one or more embodiments, the one or more spiral-shaped tubes, the cassettes, and optionally the one or more filters, are enclosed inside the reactor housing.

In one or more embodiments, the cassette comprises a plurality of openings, wherein an air flow is generated through the plurality of openings when a pressure difference is applied between an internal and external surface of the cassette, and wherein flow of air driven by said pressure difference through the plurality of openings provide a uniform cooling along the entire length of the one or more light sources in order to reach maximum UV output and ensure optimum life time of the one or more light sources.

In one or more embodiments, a space between the cassette and the one or more spiral-shaped tubes are at least partly lined with polished light reflecting aluminum reflecting light from the one or more light sources, such as reflecting at least 50% of the light back towards the one or more spiral-shaped tubes. In one or more embodiments, a space between the cassette and the one or more spiral-shaped tubes are at least partly lined with polished light reflecting aluminum reflecting at least 50% of the light from the one or more light sources back towards the one or more spiral-shaped tubes. In one or more embodiments, a space between the cassette and the one or more spiral-shaped tubes are at least partly lined with polished light reflecting aluminum reflecting at least 60% of the light from the one or more light sources back towards the one or more spiral-shaped tubes. In one or more embodiments, a space between the cassette and the one or more spiral-shaped tubes are at least partly lined with polished light reflecting aluminum reflecting at least 70% of the light from the one or more light sources back towards the one or more spiral-shaped tubes. In one or more embodiments, a space between the cassette and the one or more spiral-shaped tubes are at least partly lined with polished light reflecting aluminum reflecting at least 80% of the light from the one or more light sources back towards the one or more spiral-shaped tubes.

By reflecting light back towards the one or more spiral-shaped tubes is meant that the light hitting the polished light reflecting aluminum if reflected back, hereby preserving parts of the energy in the light, which then is reflected to the one or more spiral-shaped tubes, hereby giving a higher amount of light used to sterilize the liquid in the one or more spiral-shaped tubes. Other materials besides polished aluminum may be used, such as stainless steel, as long as the material have a high degree of reflection at the desired wavelength, thus, in one or more embodiments, a space between the cassette and the one or more spiral-shaped tubes are at least partly lined with a light reflecting material reflecting light from the one or more light sources, such as reflecting at least 50% of the light back towards the one or more spiral-shaped tubes.

In one or more embodiments, a space between two cassettes of the UV germicidal treatment system or a space between a cassette and one or more of the spiral-shaped tubes functions as a ventilation shaft used for cooling of the photo bioreactor, especially cooling the cassettes comprising the one or more light sources.

There may be a space between the two cassettes in a multiple cassette system or between a cassette and a spiral-shaped tube. Such space may be used to ventilate the air inside the space and preferably exchange the air inside the system with new air, hereby obtaining an air cooling/ventilation of the spiral-shapes tubes and/or the cassettes in the UV germicidal treatment system.

In one or more embodiments, a fluid movement through the one or more spiral-shaped tubes creates a Dean Vortex flow, laminar flow, or turbulent flow.

The present invention discloses that one of the advantages using a Dean Vortex, laminar, or turbulent flow, is that it may increase the exposure of light energy per unit volume/surface area with reduced self-shadowing effects from the opaque liquid being treated, hereby using less energy and time for treatment of the same volume.

Between the one or more spiral-shaped tubes and the one or more light sources may be located one or more filters to narrow the wavelength of the light radiated to the one or more spiral-shaped tubes to a narrower band. This will ensure an optimal wavelength for killing bacteria and viruses while avoiding oxidation of the opaque liquid food product.

By preventing light above a wavelength of 300 nm from reaching the one or more spiral-shaped tubes is meant that light above 300 nm but below 500 nm is attenuated by a substantial amount, e.g. at least a factor of 100, or a factor of 1000 or more.

In one or more embodiments, the one or more filters prevent light above a wavelength of 290 nm from reaching the one or more spiral-shaped tubes. In one or more embodiments, the one or more filters prevent light above a wavelength of 280 nm from reaching the one or more spiral-shaped tubes. In one or more embodiments, the one or more filters prevent light above a wavelength of 270 nm from reaching the one or more spiral-shaped tubes. In one or more embodiments, the one or more filters prevent light above a wavelength of 260 nm from reaching the one or more spiral-shaped tubes.

In one or more embodiments, a cross-section shape of the one or more spiral-shaped tubes is circular, hexagonal, square, triangular, or oval. The cross-section shape may have any shape, which will still maintain a large exposed outer area of the liquid product.

In one or more embodiments, the one or more spiral-shaped tubes have an inner tube diameter between 1 mm and 12 mm. In one or more embodiments, the one or more spiral-shaped tubes have an inner tube diameter between 2 mm and 11 mm. In one or more embodiments, the one or more spiral-shaped tubes have an inner tube diameter between 3 mm and 10 mm. In one or more embodiments, the one or more spiral-shaped tubes have an inner tube diameter between 4 mm and 9 mm. In one or more embodiments, the one or more spiral-shaped tubes have an inner tube diameter between 5 mm and 8 mm. In one or more embodiments, the one or more spiral-shaped tubes have an inner tube diameter between 6 mm and 8 mm.

The size of the inner diameter is a trade-off between the amounts of liquid product capable of being treated over a given time versus the exposure of light energy per unit volume/surface area. The larger the inner tube diameter is the more liquid product can pass over any given time, however, the larger the inner diameter is the smaller (relatively) the exposed area may be.

In one or more embodiments, the one or more spiral-shaped tubes have a pitch between 2 and 8 mm, wherein the pitch is the distance from centre to centre of the one or more spiral-shaped tubes after one turn/coil of the one or more spiral-shaped tubes. In one or more embodiments, the one or more spiral-shaped tubes have a pitch between 3 and 7 mm, wherein the pitch is the distance from centre to centre of the one or more spiral-shaped tubes after one turn/coil of the one or more spiral-shaped tubes. In one or more embodiments, the one or more spiral-shaped tubes have a pitch between 4 and 7 mm, wherein the pitch is the distance from centre to centre of the one or more spiral-shaped tubes after one turn/coil of the one or more spiral-shaped tubes. In one or more embodiments, the one or more spiral-shaped tubes have a pitch of 6 mm, wherein the pitch is the distance from centre to centre of the one or more spiral-shaped tubes after one turn/coil of the one or more spiral-shaped tubes. In one or more embodiments, the one or more spiral-shaped tubes have a coil angle between 1° and 6°, such as between 2° and 5°, such as between 3° and 4°, wherein the coil angle is measured between the one or more spiral-shaped tubes and a straight direction compared to the inlet end to the outlet end creating the fluidic pathway. In one or more embodiments, the one or more spiral-shaped tubes have a coil angle between 2° and 5°. In one or more embodiments, the one or more spiral-shaped tubes have a coil angle between 3° and 4°.

In one or more embodiments, the one or more spiral-shaped tubes have a coil diameter between 20 and 150 mm, wherein the coil diameter is a distance from outer end to outer end of the one or more spiral-shaped tubes after a half turn/coil of the one or more spiral-shaped tubes. That is, the coil diameter is the width of a coil created by the one or more spiral-shaped tubes.

In one or more embodiments, the one or more spiral-shaped tubes have an outer tube diameter between 2 and 8 mm. In one or more embodiments, the one or more spiral-shaped tubes have an outer tube diameter of between and 6 mm. In one or more embodiments, the one or more spiral-shaped tubes have an outer tube diameter between 3 and 7 mm. In one or more embodiments, the one or more spiral-shaped tubes have an outer tube diameter between 4 and 7 mm. In one or more embodiments, the one or more spiral-shaped tubes have an outer tube diameter of between 5 and 6 mm. In one or more embodiments, the one or more spiral-shaped tubes have an outer tube diameter of 6 mm.

In one or more embodiments, the one or more spiral-shaped tubes have a wall thickness between 0.1 and 0.4 mm. The wall thickness may also be defined as the outer tube diameter minus the inner tube diameter. In one or more embodiments, the one or more spiral-shaped tubes have a wall thickness between 0.1 and 0.3 mm. In one or more embodiments, the one or more spiral-shaped tubes have a wall thickness between 0.2 and 0.3 mm. In one or more embodiments, the one or more spiral-shaped tubes have a wall thickness between 1 and 4 mm. In one or more embodiments, the one or more spiral-shaped tubes have a wall thickness between 1 and 3 mm. In one or more embodiments, the one or more spiral-shaped tubes have a wall thickness between 2 and 3 mm.

A wall thickness between 0.1 and 4 mm is mostly used when the one or more spiral-shaped tubes are made of polymeric material, whereas the wall thickness of 1 to 4 mm is mostly used when quartz glass is used for the one or more spiral-shaped tubes. However, the wall thickness of the one or more tubes depends on the transmission percentage of the light emitted by the one or more light sources. The higher the transmission percentage, the thicker the walls can be made.

In one or more embodiments, the one or more spiral-shaped tubes are coiled around a pillar.

One advantage using a pillar to coil the one or more spiral-shaped tubes around is that a pillar stabilizes the one or more spiral-shaped tubes, if said tubes are e.g. made of a flexible material. The pillar may hence provide stabilization for the coil. Additionally, the pillar may have other advantage, e.g. helping with enhancing the amount of light radiated to the one or more spiral-shaped tubes by being e.g. reflective. In one or more embodiments, the pillar is made of a reflective material. Reflective material may be, but is not limited to, dichroic reflector material, such as aluminum, stainless steel, chromium, or silver. Reflective material may also be partly reflective materials such as Teflon materials, such as perfluoroalkoxy alkanes (PFA), polytetrafluoroethylene (PTFE), fluorinated ethylene propylene (FEP). The reflectiveness of such materials depends on the angle of the light emission on the material.

Polytetrafluoroethylene (PTFE) is a synthetic fluoropolymer of tetrafluoroethylene that has numerous applications. The best known brand name of PTFE-based formulas is Teflon. PTFE is a fluorocarbon solid, as it is a high-molecular-weight compound consisting wholly of carbon and fluorine. PTFE is hydrophobic: neither water nor water-containing substances wet PTFE, as fluorocarbons demonstrate mitigated London dispersion forces due to the high electronegativity of fluorine. PTFE has one of the lowest coefficients of friction of any solid.

Perfluoroalkoxy alkanes (PFA) are fluoropolymers. They are copolymers of tetrafluoroethylene (C2F4) and perfluoroethers (C2F3ORf, where Rf is a perfluorinated group such as e.g. trifluoromethyl (CF3)). The properties of PFA are similar to PTFE. One of the big differences is that the alkoxy substituents allow the polymer to be e.g. melt-processed. On a molecular level, PFA has a smaller chain length, and higher chain entanglement than other fluoropolymers. It also contains an oxygen atom at the branches. This results in a material that is more translucent and has improved flow, creep resistance, and thermal stability close to or exceeding PTFE.

Fluorinated ethylene propylene (FEP) is a copolymer of hexafluoropropylene and tetrafluoroethylene. It differs from the PTFE in that it is melt-processable using conventional injection molding and screw extrusion techniques. Fluorinated ethylene propylene is sold under the brand name Teflon FEP. Other brand names are Neoflon FEP or Dyneon FEP. FEP is very similar in composition to the fluoropolymers PTFE and PFA. FEP is softer than PTFE and melts around 260° C. FEP is highly transparent and resistant to sunlight.

FEP and PFA both share PTFE's useful properties of low friction and non-reactivity, but are more easily formable.

In one or more embodiments, the pillar is made of a reflective polymeric material, and in another embodiment, the pillar is covered with a metallized film. Metalized films are polymer films coated with a thin layer of metal, such as, but not limited to, aluminum. They offer the glossy metallic appearance of an aluminum foil at a reduced weight and cost.

In one or more embodiments, the one or more spiral-shaped tubes are made of a polymeric or quartz glass material being ultraviolet light transparent. However, the one or more spiral-shaped tubes can be made of any material as long as said material is more or less transparent to the light emitted by the one or more light sources. In one or more embodiments, the one or more spiral-shaped tubes are selected from fluorinated ethylene propylene (FEP), polytetrafluoroethylene (PTFE), or perfluoroalkoxy alkanes (PFA). The one or more spiral-shaped tubes may be made of any materiel with similar properties of FEP, PTFE, or PFA. In one or more embodiments, the one or more spiral-shaped tubes are from amorphous fluoropolymer (AF). The one or more spiral-shaped tubes may be made of any materiel with similar properties of AF.

Amorphous fluoropolymer (AF) is a family of amorphous fluoroplastics. These materials are similar to other amorphous polymers in optical clarity and mechanical properties, including strength. These materials are comparable to other fluoroplastics in their performance over a wide range of temperatures, in having excellent chemical resistance, and in having outstanding electrical properties. AF polymers are distinct from other fluoroplastics in that they are soluble in selected solvents, have high gas permeability, high compressibility, high creep resistance, and low thermal conductivity. AF polymers have the lowest dielectric constant of any known solid polymer. AF polymers have a low index of refraction when compared to many other known polymer.

In one or more embodiments, the one or more light sources are selected from a mercury-vapor lamp, xenon lamp, or a light emitting diode (LED). The light source of the present invention may be any light source suitable for creating light emission in the spectral wavelength area of 180 nm to 300 nm.

A mercury-vapor lamp is a gas discharge lamp that uses an electric arc through vaporized mercury to produce light. The arc discharge may be confined to a small fused quartz arc tube.

A light emitting diode (LED) is a two-lead semiconductor light source. It is a p-n junction diode that emits light when activated. When a suitable voltage is applied to the leads, electrons are able to recombine with electron holes within the device, releasing energy in the form of photons. This effect is called electroluminescence, and the color of the light (corresponding to the energy of the photon) is determined by the energy band gap of the semiconductor. LEDs are typically small (less than 1 mm) and integrated optical components may be used to shape the radiation pattern.

A xenon arc lamp is a specialized type of gas discharge lamp, an electric light that produces light by passing electricity through ionized xenon gas at high pressure. It produces a bright white light that closely mimics natural sunlight. A special kind of xenon lamp is used in automobiles. These are actually metal-halide lamps, where a xenon arc is only used during start-up.

In one or more embodiments, the one or more light sources are a metal-halide lamp. A metal-halide lamp is an electrical lamp that produces light by an electric arc through a gaseous mixture of vaporized mercury and metal halides. It is a type of high-intensity gas discharge lamp. They are similar to mercury-vapor lamps, but contain additional metal halide compounds in the quartz arc tube, which may improve the efficiency and color rendition of the light.

In one or more embodiments, the one or more light sources are selected from a light source emitting light in the ultraviolet C (UV-C) spectral wavelength area.

The ultraviolet spectra may be broken down into several smaller areas, these are: ultraviolet A (UV-A), 315-400 nm; ultraviolet B (UV-B), 280-315 nm; ultraviolet C (UV-C), 100-280 nm; near ultraviolet (N-UV), 300-400 nm; middle ultraviolet (M-UV), 200-300 nm; far ultraviolet (F-UV), 122-200 nm.

In one or more embodiments, the one or more light sources are selected from a light source emitting light in the middle ultraviolet (M-UV) spectral wavelength area.

In one or more embodiments, the one or more light sources are a low pressure germicidal lamp, such as a low-pressure mercury-vapor lamp.

A low pressure germicidal lamp may be a UV lamp that emits a significant portion of its radiative power in the UV-C band, such as a low-pressure mercury-vapor lamp or a low pressure amalgam lamp.

A low pressure amalgam lamp is a lamp doped with mercury combined with another element (often gallium) and hence is also called an amalgam lamp.

In one or more embodiments, the one or more light sources operate at a lamp temperature between 0° C. and 120° C. In one or more embodiments, the one or more light sources operate at a lamp temperature between 20° C. and 60° C. In one or more embodiments, the one or more light sources operate at a lamp temperature between 30° C. and 50° C. In one or more embodiments, the one or more light sources operate at a lamp temperature of 40° C.

The present invention discloses that one of the advantages by utilizing a light source with a lower lamp temperature may be that less heat is transferred from the light source to the opaque liquid product. This may yield a lower requirement for cooling of the liquid product during the germicidal treatment.

Cold pasteurization may be partial sterilization of a substance and especially a liquid in a process where heat is evaded as the main eradication of objectionable organisms without major chemical alteration of the substance. With evaded is not meant excluded but reduced. The present invention discloses that one of the advantages of using light radiation as a means for cold pasteurization is that it is a very energy efficient way for partial sterilization.

In one or more embodiments, a biological contaminant is inactivated or reduced by an order of at least 2-Log10. A biological contaminant may be e.g., bacteria, spores, mold, or virus, such as bacteria, spores, mold, or virus selected from Campylobacter jejuni, Shigella, Coxiella burnetii, Escherichia coli, Listeria monocytogenes, Mycobacterium bovis, Mycobacterium tuberculosis, Mycobacterium paratuberculosis, Salmonella spp., Yersinia enterocolitica, Brucella spp., Staphylococcus spp., Lactobacillus casei, Mycobacterium avium subspecies, Staphylococcus aureus, Streptococcus spp., Enterococcus spp., or Entrerobacter spp.

In one or more embodiments, a biological contaminant is inactivated or reduced by an order of at least 3-Log10. In one or more embodiments, a biological contaminant is inactivated or reduced by an order of at least 4-Log10. In one or more embodiments, a biological contaminant is inactivated or reduced by an order of at least 5-Log10. In one or more embodiments, a biological contaminant is inactivated or reduced by an order of at least 6-Log10.

With killing is meant reducing the amount of active or living microorganisms. Microorganisms found in liquid products, such as liquid food products, may be present due to contamination during the process of said liquid product. Common bacteria contamination of e.g. dairy products may be e.g., Lactobacillus casei, Escherichia coli, Listeria monocytogenes, Salmonella spp., Mycobacterium avium subspecies paratuberculosis (MAP), Staphylococcus aureus, or Streptococcus spp.

In one or more embodiments, the opaque liquid products are selected from liquid dairy products, such as raw milk, cream, milk, or rennet; juice, such as orange-, apple-, tomato-, or pineapple juice; coffee; tea; soya; soylent; soda; broth; soup; beer; smoothies; protein shake; liquid meal-replacement; wine; mayonnaise; ketchup; syrup; honey; egg, such as egg yolk or egg white; blood, such as whole blood or plasma; or opaque water, such as brine, pickle, or opaque processing water.

When describing the embodiments of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The invention will hereafter be described by way of the following non-limiting items.
1. A UV germicidal treatment system for treatment of opaque liquids, wherein the UV germicidal treatment system comprises:
   one or more spiral-shaped tubes extending from an inlet end to an outlet end creating a fluidic pathway;
   one or more means for controlling a flowrate of the opaque liquids through the fluidic pathway when the UV germicidal treatment system is in use;
   one or more UV light sources illuminating the one or more spiral-shaped tubes, wherein the one or more light sources emit light in a wavelength range between 180-300 nm; and
   a surveillance system configured for monitoring and controlling parameters of the UV germicidal treatment system for optimizing the germicidal treatment of the opaque liquids when the UV germicidal treatment system is in use;
wherein the surveillance system comprises one or more UV light sensors configured to monitor an UV output related characteristics of the one or more UV light sources and provide an output accordingly, and wherein the one or more UV light sensors are positioned in the UV germicidal treatment system such that the one or more UV light sensors directly or indirectly measures an UV light intensity substantially proportional to the UV light intensity illuminating the one or more spiral-shaped tubes when the UV germicidal treatment system is in use;
wherein the surveillance system further comprises at least one flow sensor, wherein the flow sensor is configured to monitor a flow related characteristics of the opaque liquid within the fluidic pathway when the UV germicidal treatment system is in use and provide an output accordingly, and wherein the flow sensor is positioned at or in the inlet end or at or in the outlet end of the fluidic pathway; and
wherein the surveillance system further comprises a controller configured for:
   receiving a first input relating to the UV output related characteristics of the one or more UV light sources;
   receiving a second input relating to the flow related characteristics of the opaque liquid within the fluidic pathway;
   determining an UV treatment condition for the UV germicidal treatment system based on the received first and second input and the opaque liquids to be treated; and
   controlling the one or more UV light sources and/or the one or more means for controlling the flowrate based on the determined UV treatment condition.
2. The UV germicidal treatment system according to item 1, wherein the controller is configured for controlling the one or more UV light sources to keep the UV output of the one or more UV light sources on a predefined value.
3. The UV germicidal treatment system according to any of items 1 or 2, wherein the one or more means for controlling the flowrate is selected from one or more pumps, one or more valves, one or more pressurized tank systems, or a combination hereof.
4. The UV germicidal treatment system according to any of the proceeding items, wherein the at least one flow sensor is selected from a flowrate sensor, a pressure sensor, or combinations hereof.
5. The UV germicidal treatment system according to any of the proceeding items, wherein the controller is configured with a predefined minimum value and a predefined maximum value for determining the UV treatment condition, and wherein the controller is configured to determine the UV treatment condition based on the received first and second input, such that the UV treatment condition is within the predefined minimum and maximum value.
6. The UV germicidal treatment system according to any of the proceeding items, wherein the controller is configured to control both the one or more UV light sources and the one or more means for controlling the flowrate, and wherein the controller is further configured to control the one or more UV light sources and/or the one or more means for controlling the flowrate based on the determined UV treatment condition.
7. The UV germicidal treatment system according to any of the proceeding items, wherein the UV germicidal treatment system is for avoiding overexposure of opaque liquids to be treated.
8. The UV germicidal treatment system according to any of items 1-6, wherein the UV germicidal treatment system is for reducing the amount of energy used by the UV germicidal treatment during treatment of opaque liquids.
9. The UV germicidal treatment system according to any of the proceeding items, further comprising one or more valves configured to change the fluidic pathway within the one or more spiral-shaped tubed and one or more additional internal UV light sensors positioned inside the fluidic pathway of the one or more spiral-shaped tubes,
wherein the additional internal UV light sensors are configured to monitor an UV output related to opaque characteristics of the liquid within the fluidic pathway during use and provide an output accordingly, and
wherein the controller is further configured for:
   receiving a third input relating to the UV output related to opaque characteristics of the liquid,
   determining if the opaque characteristics of the liquid changes, and
   controlling the one or more valves such as changing the fluidic pathway of the one or more spiral-shaped tubes within the UV germicidal treatment system if the opaque characteristics of the liquid changes.
10. The UV germicidal treatment system according to any of the proceeding items, wherein the controller is further configured for determining a first ratio between the UV output related to opaque characteristics of the liquid within the fluidic pathway and the UV output related characteristics of the one or more UV light sources at an initial state of the UV germicidal treatment system in which the spiral-shaped tubes are clean and in which a standard liquid transparent to UV, such as water, is provided within the fluidic pathway, and wherein this first ratio is defined as a clean state ratio of the UV germicidal treatment system.
11. The UV germicidal treatment system according to any of the proceeding items, wherein the UV germicidal treatment system is further configured for a predefined period of UV germicidal treatment of opaque liquids followed by a predefined period of flushing the UV germicidal treatment system with standard liquid transparent to UV, such as water, wherein the controller is further configured for determining a second ratio between the UV output related to opaque characteristics of the liquid within the fluidic pathway and the UV output related characteristics of the one or more UV light sources after the predefined period of flushing when the standard liquid transparent to UV, such as water, is provided within the fluidic pathway, and comparing the ratio with the clean state ratio for determining and quantify a rate of fouling or determine a return to a clean state.
12. The UV germicidal treatment system according to any of the proceeding items, wherein the controller is configured for providing an alarm to a user if the second input relating to the flow related characteristics of the opaque liquid within the fluidic pathway is below a predefined minimum value.
13. The UV germicidal treatment system according to any of the proceeding items, wherein the at least one flow sensor is at least two flow sensors, wherein at least one flow sensor is selected from a pressure sensor and at least one other flow sensor is selected from a flowrate sensor.
14. The UV germicidal treatment system according to any of the proceeding items, wherein the at least one flow sensor is a pressure sensor, wherein the flow sensor is positioned at or in the inlet end of the fluidic pathway, and wherein the controller is configured for providing an alarm to a user if the second input relating to the flow related characteristics of the opaque liquid within the fluidic pathway is below a predefined minimum value.
15. The UV germicidal treatment system according to any of the proceeding items, wherein the at least one flow sensor is at least two flow sensors, wherein at least one flow sensor is positioned at or in the inlet end of the fluidic pathway and at least one other flow sensor is positioned at or in the outlet end of the fluidic pathway.
16. The UV germicidal treatment system according to any of the proceeding items, wherein at least one flow sensor is positioned at or in the outlet end of the fluidic pathway.
17. The UV germicidal treatment system according to any of the proceeding items, wherein the controller is configured for:
   receiving an input from the at least two flow sensors,
   determining a ratio between the input from the at least two flow sensors, and
   providing an alarm to a user if the ratio between the input from the at least two flow sensors is above a predefined maximum ratio or below a predefined minimum ratio.
18. The UV germicidal treatment system according to any of the proceeding items, wherein the at least one flow sensor comprises at least two flow sensors both positioned at or in the inlet end or at or in the outlet end of the fluidic pathway and wherein the controller is configured to continuously compared deviations in flow related output of the two flow sensors.
19. The UV germicidal treatment system according to any of the proceeding items, wherein the controller is configured for increasing or decreasing the light emitted by the one or more UV light sources illuminating the one or more spiral-shaped tubes if the controller determines drop or increase in the second input relating to the flow related characteristics of the opaque liquid within the fluidic pathway.
20. The UV germicidal treatment system according to any of the proceeding items, wherein the UV germicidal treatment system further comprises one or more filters positioned between the one or more light sources and the one or more spiral-shaped tubes, wherein the one or more filters prevent light above a wavelength of 300 nm from reaching the one or more spiral-shaped tubes.
21. The UV germicidal treatment system according to any of the proceeding items, wherein the system further comprises an adaptive cooling system comprising one or more blowing units for driving an airflow through the UV germicidal treatment system.
22. The UV germicidal treatment system according to item 21, wherein the one or more blowing units are configured for driving the airflow through the UV germicidal treatment system by creating a negative pressure inside the UV germicidal treatment system.
23. The UV germicidal treatment system according to any of items 21-22, wherein the one or more blowing units are configured for driving the airflow through the UV germicidal treatment system by creating a positive pressure inside the UV germicidal treatment system.
24. The UV germicidal treatment system according to any of items 21-23, wherein the adaptive cooling system further comprises one or more temperature sensors.
25. The UV germicidal treatment system according to item 24, wherein the one or more temperature sensors are positioned at or in an air outlet or at or in an air outlet of the UV germicidal treatment system.
26. The UV germicidal treatment system according to any of items 21-25, wherein the controller is configured for controlling the adaptive cooling system based on effect, such as Watt used, of the one or more UV light sources illuminating the one or more spiral-shaped tubes.
27. The UV germicidal treatment system according to any of items 24-26, wherein the controller is configured for controlling the adaptive cooling system based on a received input from the one or more temperature sensors.
28. The UV germicidal treatment system according to any of items 24-27, wherein the one or more temperature sensors are at least two temperature sensors positioned at different positions within the UV germicidal treatment system, and wherein the controller is configured for controlling the adaptive cooling system based on a difference between a received input from one of the at least two temperature sensors and a received input from another of the at least two temperature sensors.
29. The UV germicidal treatment system according to any of items 24-28, wherein the controller is configured for controlling the adaptive cooling system based on a received input from one or more temperature sensors positioned at or in an air outlet of the UV germicidal treatment system.
30. The UV germicidal treatment system according to any of items 24-29, wherein the controller is configured for controlling the adaptive cooling system based on a received input from one or more temperature sensors positioned at or in an air inlet of the UV germicidal treatment system.
31. The UV germicidal treatment system according to any of items 24-30, wherein the controller is configured for providing an alarm to a user if the controller receives an input from that the one or more temperature sensors above a predefined value.
32. The UV germicidal treatment system according to any of items 24-31, wherein the controller is configured for providing an alarm to a user if the controller receives an input from that the one or more temperature sensors above a predefined value and receives a maximum capacity input from the one or more blowing units.
33. The UV germicidal treatment system according to any of the proceeding items, wherein the UV germicidal treatment system further comprises a first cassette mounting frame and at least two cassettes extending from a first end to a second end;
   wherein the cassette mounting frame comprises cassette receiving openings into which each of the cassettes are removable mounted;
   wherein each cassette comprises the one or more light sources illuminating the one or more spiral-shaped tubes; and
   wherein one or more of the one or more spiral-shaped tubes are positioned between two of at least two cassettes.
34. The UV germicidal treatment system according to item 33, wherein each cassette also comprises one or more of the one or more filters, such that the one or more filters are positioned between the one or more light sources and the one or more spiral-shaped tubes.
35. The UV germicidal treatment system according to any of items 33-34, wherein the one or more of the spiral-shaped tubes are grouped in sets of two, such as sets of three, positioned in a configuration alternating between a set of one or more of the spiral-shaped tubes and a cassette.
36. The UV germicidal treatment system according to any of items 33-35, wherein the UV germicidal treatment system further comprises a first ventilation chamber positioned at the first end of the one or more cassettes.
37. The UV germicidal treatment system according to item 36, wherein the UV germicidal treatment system further comprises a second ventilation chamber positioned at the second end of the one or more cassettes.
38. The UV germicidal treatment system according to any of items 36-37, wherein the ventilation chamber pulls air out of the cassette or at the ventilation chamber air flows into the cassette.
39. The UV germicidal treatment system according to any of items 36-38, wherein the ventilation chamber pulls air out of the cassette at both ends.
40. The UV germicidal treatment system according to any of items 36-38, wherein at the ventilation chamber air flows into the cassette at both ends.
41. The UV germicidal treatment system according to any of items 37-38, wherein the ventilation chamber pulls air out of the cassette at one end and air flows into the cassette at the other end.
42. The UV germicidal treatment system according to any of items 33-41, wherein each of the cassettes further comprises air intake openings for allowing air to flow into the cassette.
43. The UV germicidal treatment system according to any of items 33-42, wherein each of the cassettes further comprises a cassette frame with openings, wherein a first set of openings are covered by glass, such as quartz glass, through which light from the light sources can illuminate the one or more of the spiral-shaped tubes.
44. The UV germicidal treatment system according to any of items 33-43, wherein each of the cassettes further comprises a cassette frame with openings, wherein a second set of openings are adapted for facilitating internal air movement inside the cassette.
45. The UV germicidal treatment system according to any of items 33-44, wherein the cassette comprises a plurality of openings, wherein an air flow is generated through the plurality of openings when a pressure difference is applied between an internal and external surface of the cassette, and wherein flow of air driven by said pressure difference through the plurality of openings provide a uniform cooling along the entire length of the one or more light sources in order to reach maximum UV output and ensure optimum life time of the one or more light sources.
46. The UV germicidal treatment system according to any of items 33-45, wherein a space between two cassettes of the UV germicidal treatment system or a space between a cassette and one or more of the spiral-shaped tubes functions as a ventilation shaft used for cooling of the UV germicidal treatment system, especially cooling the cassettes comprising the one or more light sources.
47. The UV germicidal treatment system according to any of the proceeding items, the one or more light sources operate at a lamp temperature between 0° C. and 120° C., such as between 20° C. and 100° C., such as between 40° C. and 100° C., such as between 60° C. and 100° C., or such as between 80° C. and 100° C.
48. The UV germicidal treatment system according to item 22 or 34 wherein the one or more filters are selected from band-pass filters, notch filters, or a combination of both.
49. The UV germicidal treatment system according to any of the proceeding items, wherein the UV germicidal treatment system further comprises a reactor housing.
50. The UV germicidal treatment system according to item 49, wherein the one or more spiral-shaped tubes, the cassettes, and optionally the one or more filters, are enclosed inside the reactor housing.
51. Use of a UV germicidal treatment system according to any of the proceeding items for cold pasteurization of opaque liquid products, such as an opaque liquid food product.
52. Use of a UV germicidal treatment system according to any of items 1-50 for killing microorganisms, such as bacteria, mold, spores, or virus, in opaque liquid products, such as an opaque liquid food product.
53. Use of a UV germicidal treatment system according to any of items 51-52, wherein the opaque liquid products are selected from liquid dairy products, such as raw milk, cream, milk, or rennet; juice, such as orange-, apple-, tomato-, or pineapple juice; coffee; tea; soya; soylent; soda; broth; soup; beer; smoothies; protein shake; liquid meal-replacement; wine; mayonnaise; ketchup; syrup; honey; egg, such as egg yolk or egg white; blood, such as whole blood or plasma; or opaque water, such as brine, pickle, or opaque processing water.
54. A surveillance system configured for monitoring and controlling parameters of a UV germicidal treatment system for optimizing the germicidal treatment of the opaque liquids when the UV germicidal treatment system is in use;
wherein the surveillance system comprises one or more UV light sensors configured to monitor an UV output related characteristics of one or more UV light sources and provide an output accordingly;
wherein the surveillance system further comprises at least one flow sensor, wherein the flow sensor is configured to monitor a flow related characteristics of an opaque liquid within a fluidic pathway when the UV germicidal treatment system is in use and provide and output accordingly; and
wherein the surveillance system further comprises a controller configured for:
   receiving a first input relating to the UV output related characteristics of the one or more UV light sources;
   receiving a second input relating to the flow related characteristics of the opaque liquid within the fluidic pathway;
   determining an UV treatment condition for the UV germicidal treatment system based on the received first and second input and the opaque liquids to be treated; and
   controlling the one or more UV light sources and/or the one or more means for controlling the flowrate based on the determined UV treatment condition.
55. Use of a surveillance system according to item 54. for optimizing germicidal treatment of opaque liquids treated in an UV germicidal treatment system.
56. Method for optimizing germicidal treatment of opaque liquids in an UV germicidal treatment system,
wherein the method comprises the following steps:
   providing the opaque liquid through a fluidic pathway in the UV germicidal treatment system;
   controlling a flow rate of the opaque liquids through the fluidic pathway via one or more means for controlling the flow rate;
   illuminating the fluidic pathway by emitting light in a wavelength range between 180-300 nm from one or more UV light sources;
   monitor and control parameters of the UV germicidal treatment system via a surveillance system comprising one or more UV light sensors, at least one flow sensor, and a controller, wherein the UV light sensors monitor an UV output related characteristics of the one or more UV light sources and provide an output accordingly and the at least one flow sensor monitor a flow related characteristics of the opaque liquid within the fluidic pathway and provide an output accordingly, wherein the monitor and control comprises the following steps;
   provide a first input relating to the UV output related characteristics of the one or more UV light sources to the controller;
   provide a second input relating to the flow related characteristics of the opaque liquid within the fluidic pathway to the controller;
   determine an UV treatment condition for the UV germicidal treatment system based on the received first and second input and the opaque liquids to be treated; and
   control the one or more UV light sources and/or the one or more means for controlling the flowrate based on the determined UV treatment condition.

### BRIEF DESCRIPTION OF THE DRAWINGS

FIG. 1 shows a flow chart describing one embodiment of the present invention.

FIG. 2 shows schematically illustrated air flow of the system in an embodiment of the present invention as a front view showing multiple cassettes with light sources and multiple spiral-shaped tubes.

FIG. 3 shows a zoomed in illustration of the embodiment of FIG. 2.

FIG. 4 shows a cut-out view of an embodiment of the present invention showing a cassette with a light source, a spiral-shaped tube, and two UV light sensors.

### DETAILED DESCRIPTION OF THE DRAWINGS

The present disclosure will now be described with reference to the accompanying drawings, in which example embodiments of the disclosure are shown. The disclosure may, however, be embodied in other forms and should not be construed as limited to the herein disclosed embodiments. The disclosed embodiments are provided to fully convey the scope of the disclosure to the skilled person.

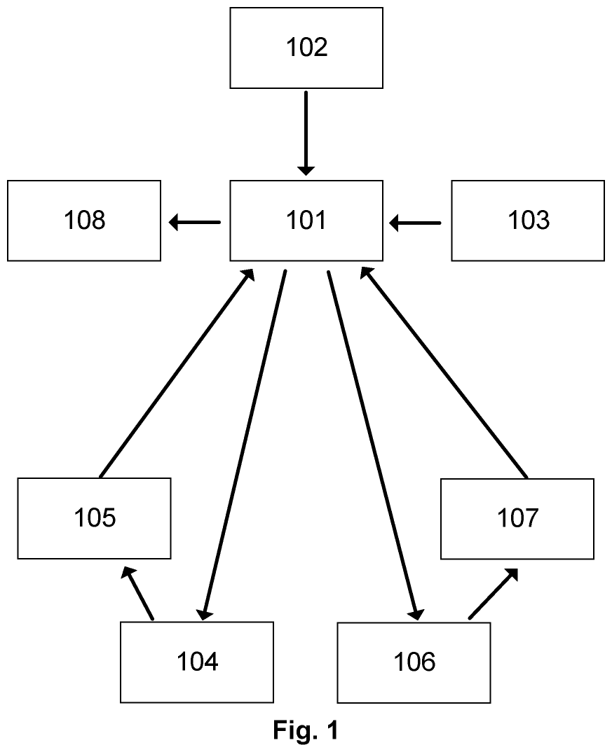

FIG. 1 shows a flow chart describing one embodiment of the present invention. The embodiment as shown in FIG. 1 shows a process for the controller to control or monitor the system based on the configurations and the inputs received. The controller 101 is configured with a recipe input 102 from the user controlling the system. The recipe input 102 can be a specific recipe defined in terms of required UV intensity and required minimum and maximum flow. When the system starts the controller 101 receives an input from the flow sensor 103. This input specifies or is basis for a calculation of flow measured. The controller 101 is then able to determine if the flow is within the range defined in the recipe input 102.

The system of this particular embodiment further comprises one or more fans 104, one or more temperature sensors 105, one or more UV light sources 106, and one or more UV light sensors 107.

The UV intensity is defined by the power level of the UV light source 106 and measured by the UV light sensor 107. The controller 101 receives an input from the UV light sensor 107 and is able to adjust the UV light source 106 e.g. by adjusting the power to keep the value set in the recipe input 102, hence, the controller 101 may based on the input from the UV light sensor 107 control the UV light source 106 as shown by the arrows.

The controller 101 is further able to control the cooling air supply from the one or more fans 104 to maintain the correct cooling level of the UV light source 106. The temperature of cooling air depends on the UV light source 106 power level, the inlet air temperature and the cooling fan speed of the one or more fans 104. The controller 101 receives an input from the one or more temperature sensors 105 about the cooling air temperature and is able to adjusts fan speed of the one or more fans 104 based on this input to achieve an optimum cooling conditions required for optimum UV light source 106 performance.

The controller is further able to provide an I/O signal 108 to inform a user about the status of the UV germicidal treatment and/or the status of the system.

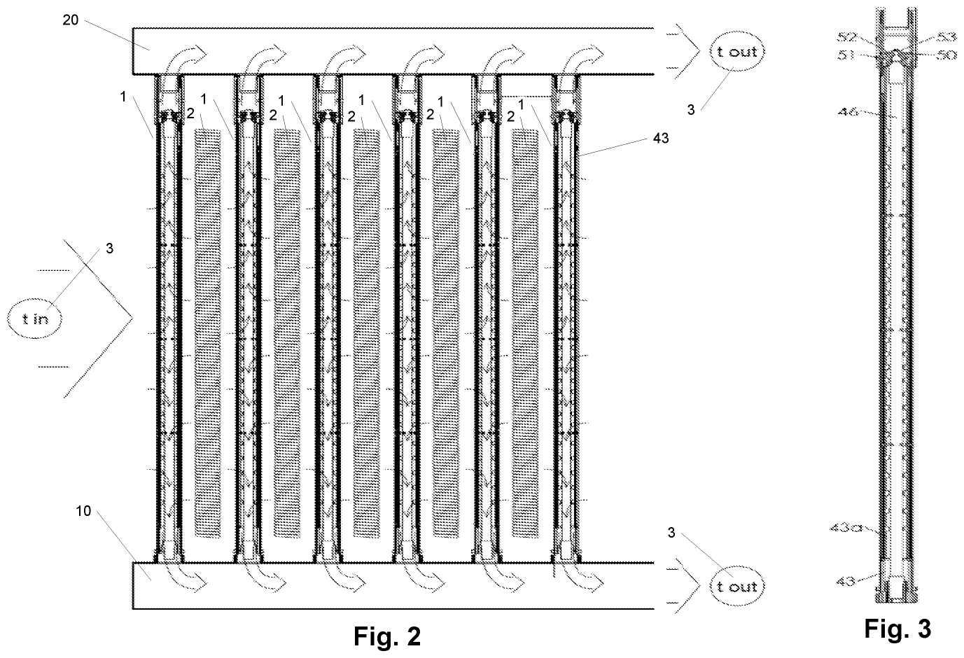

FIG. 2 shows one embodiment of the present invention. In the embodiment, a cassette 1 is placed parallel to a spiral-shaped tube 2.

The cassettes 1 are mounted into a bottom ventilation chamber/manifold 10 through which air is being sucked out at the ends (airflow is marked with arrows). A cassette mounting frame may be used to hold the cassettes in place. The embodiment further comprises a top ventilation chamber/manifold 20 through which air is being sucked out at the ends (airflow is marked with arrows). A gasket may be used between the cassette 1 and the top ventilation chamber 20 to create a seal.

The bottom ventilation chamber 10 has rectangular holes where cassettes are joined using gaskets to create a seal. Air can be sucked out at the ends as shown in the figure. A cassette mounting frame may be welded to the bottom ventilation chamber 10 to keep the cassettes in place.

The cassette 1 further comprises a sheet metal part 43 with multiple cut-outs for air intake into the cassette (airflow is marked with arrows) and which aid in blocking UV light hereby evading UV light in escaping the cassette 1.

The embodiment further illustrates where temperature sensors 3 may be placed in the system. As marked on the figure one temperature sensor 3 may be used to measure the temperature of air when entering the system (t in) and one or two temperature sensors 3 may be used to measure the temperature of air when exiting the system (t out) through the bottom and top ventilation chambers 10, 20.

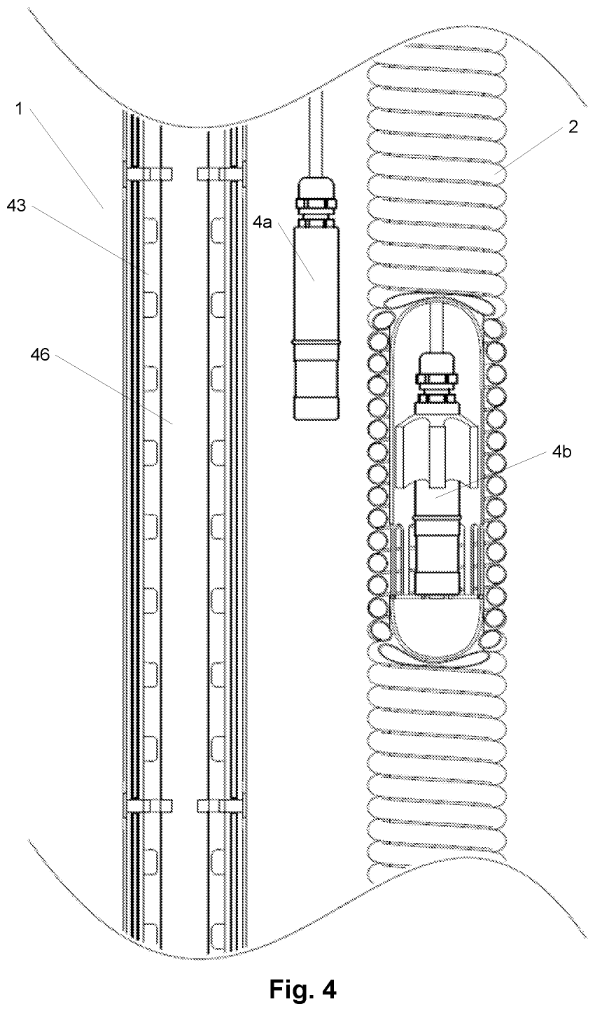

FIG. 3 shows the section of FIG. 2. The cassette 1 comprises a sheet metal part 43 with multiple cut-outs for air intake into the cassette. The sheet metal part 43 is further used for blocking UV light hereby preventing it from escaping the cassette. Another sheet metal part 43a comprises cut-outs wherein the size of the cut-outs are based on calculation. The sheet metal part 43a is used for evenly distributed cooling of the light sources 46. The cut-outs or the sheet metal part 43a are not aligning with the cut-outs of the sheet metal part 43, hereby allowing air movement through the cut-outs but blocking UV light hereby preventing light from escaping the cassette. A further sheet metal part 50 is used to hold a milled plastic part that is holding a ceramic light source pin connector 53, while a locking part 52 is locking the milled plastic part 51 in place.

FIG. 4 shows one embodiment of the present invention. The figure shows parts of the UV germicidal treatment system being cut away, especially parts of the spiral-shaped tube 2 has been removed to make an internal UV light sensor 4b visible. In the embodiment, a cassette 1 is placed parallel to a spiral-shaped tube 2. Further shown and similar to the embodiment of FIGS. 2 and 3 is shown the sheet metal parts 43, and the light sources 46. The embodiment further illustrates how two UV light sensors 4a, 4b are positioned. The UV light sensors shown are an external UV light sensor 4a and an internal UV light sensor 4b.

The external UV light sensor 4a is positioned somewhere in the UV germicidal treatment system between the cassette 1 comprising the light source 46 and the spiral-shaped tube 2, such that it is able to measure the amount/intensity of UV light from the light source 46 reaching the spiral-shaped tube 2 but without blocking the light emitted from the light source 46. If the light source 46 is not placed in a cassette 1, the external UV light sensor 4a should still be positioned somewhere in the UV germicidal treatment system between the light source 46 and the spiral-shaped tube 2.

The internal UV light sensor 4b is positioned somewhere in the UV germicidal treatment system such that the light emitted from the light source 46 is passing through the spiral-shaped tube 2 prior to reaching the internal UV light sensor 4b. In the present embodiment, the internal UV light sensor 4b is positioned in the centre of (inside) the spiral-shaped tube 2, such that it measures the amount/intensity of UV light from the light source 46 passing through the spiral-shaped tube 2 and passing through the liquid, when the system is in use.

### REFERENCES

1—Cassette
2—Spiral-shaped tube
3—Temperature sensor
4a—External UV light sensor
4b—Internal UV light sensor
10—Bottom ventilation chamber/manifold
20—Top ventilation chamber/manifold
43, 43a—Sheet metal part with cut-outs
46—Light source
50—Sheet metal part
51—Milled plastic part
52—Locking part
53—Ceramic light source pin connector
101—Controller
102—Recipe input
103—Flow sensor
104—Fan
105—Temperature sensor
106—UV light source
107—UV light sensor
108—I/O signal

1. A UV germicidal treatment system for treatment of opaque liquids, wherein the UV germicidal treatment system comprises:
one or more spiral-shaped tubes, each of the one or more spiral-shaped tubes extending from an inlet end to an outlet end creating a fluidic pathway;
one or more flow rate controllers that control a flow rate of opaque liquids through the fluidic pathway of each of the one or more spiral-shaped tubes;
one or more UV light sources illuminating the one or more spiral-shaped tubes, wherein the one or more UV light sources emit light in a wavelength range between 180-300 nm; and
a surveillance system configured for monitoring and controlling parameters of the UV germicidal treatment system for optimizing the germicidal treatment of the opaque liquids, wherein the surveillance system comprises:
one or more UV light sensors configured to monitor an UV output related characteristics of the one or more UV light sources and provide an output accordingly, and wherein the one or more UV light sensors are positioned in the UV germicidal treatment system such that the one or more UV light sensors directly or indirectly measure a UV light intensity substantially proportional to the UV light intensity illuminating the one or more spiral-shaped tubes;
at least one flow sensor, wherein the at least one flow sensor is configured to monitor a flow related characteristics of the opaque liquid within the fluidic pathway of each of the one or more spiral-shaped tubes and provide an output accordingly, and wherein the flow sensor is positioned at or in the inlet end or at or in the outlet end of the fluidic pathway of each of the one or more spiral-shaped tubes; and
a controller configured for:
receiving a first input relating to the UV output related characteristics of the one or more UV light sources;
receiving a second input relating to the flow related characteristics of the opaque liquids within the fluidic pathway of each of the one or more spiral-shaped tubes;
determining a UV treatment condition for the UV germicidal treatment system based on the received first input and the received second input and the opaque liquids within the fluidic pathway of each of the one or more spiral-shaped tubes to be treated; and
controlling the one or more UV light sources and/or the one or more flow rate controllers that control the flowrate based on the determined UV treatment condition.

2. The UV germicidal treatment system according to claim 1, wherein the controller is configured with a predefined minimum value and a predefined maximum value for determining the UV treatment condition, and wherein the controller is configured to determine the UV treatment condition based on the received first input and the second input, such that the UV treatment condition is within the predefined minimum and the predefined maximum value.

3. The UV germicidal treatment system according to claim 1, wherein the controller is configured to control both the one or more UV light sources and the one or more flow rate controllers that control the flow rate, and wherein the controller is further configured to control the one or more UV light sources and/or the one or more flow rate controllers that control the flow rate based on the determined UV treatment condition.

4. The UV germicidal treatment system according to claim 1, further comprising one or more valves configured to change the fluidic pathway within each of the one or more spiral-shaped tubes and one or more additional internal UV light sensors positioned inside the fluidic pathway of each of the one or more spiral-shaped tubes, wherein the additional internal UV light sensors are configured to monitor a UV output related to opaque characteristics of the opaque liquid within the fluidic pathway of each of the one or more spiral-shaped tubes during use and provide an output accordingly, and
wherein the controller is further configured for:
receiving a third input relating to the UV output related to opaque characteristics of the opaque liquid of each of the one or more spiral-shaped tubes,
determining if the opaque characteristics of the opaque liquid of each of the one or more spiral-shaped tubes changes, and
controlling the one or more valves such as changing the fluidic pathway of each of the one or more spiral-shaped tubes within the UV germicidal treatment system if the opaque characteristics of the opaque liquid of each of the one or more spiral-shaped tubes changes.

5. The UV germicidal treatment system according to claim 1, wherein the UV germicidal treatment system is further configured for a predefined period of UV germicidal treatment of opaque liquids of each of the one or more spiral-shaped tubes followed by a predefined period of flushing the UV germicidal treatment system with standard liquid transparent to UV, wherein the controller is further configured for determining a ratio between the UV output related to opaque characteristics of the opaque liquid within the fluidic pathway of each of the one or more spiral-shaped tubes and the UV output related characteristics of the one or more UV light sources after the predefined period of flushing when the standard liquid transparent to UV, is provided within the fluidic pathway of each of the one or more spiral-shaped tubes, and comparing the ratio with a clean state ratio for determining and quantify a rate of fouling or determine a return to the clean state.

6. The UV germicidal treatment system according to claim 1, wherein the system further comprises an adaptive cooling system comprising one or more blowing units for driving an airflow through the UV germicidal treatment system;
wherein the one or more blowing units are configured for driving the airflow through the UV germicidal treatment system by creating a negative or positive pressure inside the UV germicidal treatment system;
wherein the adaptive cooling system further comprises one or more temperature sensors;
wherein the controller is configured for controlling the adaptive cooling system based on effect of the one or more UV light sources illuminating the one or more spiral-shaped tubes or wherein the controller is configured for controlling the adaptive cooling system based on a received input from the one or more temperature sensors.

7. The UV germicidal treatment system according to claim 1, wherein the UV germicidal treatment system further comprises a first cassette mounting frame and at least two cassettes extending from a first end to a second end;
wherein the cassette mounting frame comprises cassette receiving openings into which each of the at least two cassettes are removable mounted;
wherein each of the at least two cassettes comprises the one or more light sources illuminating the one or more spiral-shaped tubes; and
wherein at least one of the one or more spiral-shaped tubes is positioned between two of the at least two cassettes;
wherein the UV germicidal treatment system further comprises a first ventilation chamber positioned at the first end of the at least two cassettes;
wherein the UV germicidal treatment system further comprises a second ventilation chamber positioned at the second end of the at least two cassettes;
wherein the first ventilation chamber and/or the second ventilation chamber pulls air out of the at least two cassettes or at the first ventilation chamber and/or the second ventilation chamber air flows into the at least two cassettes; and
wherein each of the at least two cassettes comprises a plurality of openings, wherein an air flow is generated through the plurality of openings when a pressure difference is applied between an internal surface and an external surface, and wherein flow of air driven by the pressure difference through the plurality of openings provide a uniform cooling along the entire length of the one or more light sources in order to reach a maximum UV output and ensure optimum life time of the one or more light sources.

8. A method of cold pasteurization of opaque liquids using Use of a UV germicidal treatment system comprising:
one or more spiral-shaped tubes, each of the one or more spiral-shaped tubes extending from an inlet end to an outlet end creating a fluidic pathway;
one or more flow rate controllers that control a flow rate of opaque liquids through the fluidic pathway of each of the one or more spiral-shaped tubes;
one or more UV light sources illuminating the one or more spiral-shaped tubes, wherein the one or more UV light sources emit light in a wavelength range between 180-300 nm; and
a surveillance system configured for monitoring and controlling parameters of the UV germicidal treatment system for optimizing the germicidal treatment of the opaque liquids, wherein the surveillance system comprises:
one or more UV light sensors configured to monitor an UV output related characteristics of the one or more UV light sources and provide an output accordingly, and wherein the one or more UV light sensors are positioned in the UV germicidal treatment system such that the one or more UV light sensors directly or indirectly measure a UV light intensity substantially proportional to the UV light intensity illuminating the one or more spiral-shaped tubes;
at least one flow sensor, wherein the at least one flow sensor is configured to monitor a flow related characteristics of the opaque liquid within the fluidic pathway of each of the one or more spiral-shaped tubes and provide an output accordingly, and wherein the flow sensor is positioned at or in the inlet end or at or in the outlet end of the fluidic pathway of each of the one or more spiral-shaped tubes; and
a controller configured for:
receiving a first input relating to the UV output related characteristics of the one or more UV light sources;
receiving a second input relating to the flow related characteristics of the opaque liquids within the fluidic pathway of each of the one or more spiral-shaped tubes;
determining a UV treatment condition for the UV germicidal treatment system based on the received first input and the received second input and the opaque liquids within the fluidic pathway of each of the one or more spiral-shaped tubes to be treated; and
controlling the one or more UV light sources and/or the one or more flow rate controllers that control the flowrate based on the determined UV treatment condition;
the method comprising:
performing cold pasterization of the opaque liquids of the one or more spiral-shaped tubes.

9. A method of cold pasteurization of opaque liquids using a UV germicidal treatment system comprising:
one or more spiral-shaped tubes, each of the one or more spiral-shaped tubes extending from an inlet end to an outlet end creating a fluidic pathway;
one or more flow rate controllers that control a flow rate of opaque liquids through the fluidic pathway of each of the one or more spiral-shaped tubes;
one or more UV light sources illuminating the one or more spiral-shaped tubes, wherein the one or more UV light sources emit light in a wavelength range between 180-300 nm; and
a surveillance system configured for monitoring and controlling parameters of the UV germicidal treatment system for optimizing the germicidal treatment of the opaque liquids, wherein the surveillance system comprises:
one or more UV light sensors configured to monitor an UV output related characteristics of the one or more UV light sources and provide an output accordingly, and wherein the one or more UV light sensors are positioned in the UV germicidal treatment system such that the one or more UV light sensors directly or indirectly measure a UV light intensity substantially proportional to the UV light intensity illuminating the one or more spiral-shaped tubes;
at least one flow sensor, wherein the at least one flow sensor is configured to monitor a flow related characteristics of the opaque liquid within the fluidic pathway of each of the one or more spiral-shaped tubes and provide an output accordingly, and wherein the flow sensor is positioned at or in the inlet end or at or in the outlet end of the fluidic pathway of each of the one or more spiral-shaped tubes; and
a controller configured for:
receiving a first input relating to the UV output related characteristics of the one or more UV light sources;
receiving a second input relating to the flow related characteristics of the opaque liquids within the fluidic pathway of each of the one or more spiral-shaped tubes;
determining a UV treatment condition for the UV germicidal treatment system based on the received first input and the received second input and the opaque liquids within the fluidic pathway of each of the one or more spiral-shaped tubes to be treated; and
controlling the one or more UV light sources and/or the one or more flow rate controllers that control the flowrate based on the determined UV treatment condition;
the method comprising:
killing microorganisms of the opaques liquids of the one or more spiral-shaped tubes.

10. A surveillance system configured for monitoring and controlling parameters of a UV germicidal treatment system for treatment of an opaque liquid when the UV germicidal treatment system is in use, wherein the surveillance system comprises:
one or more UV light sensors configured to monitor an UV output related characteristics of one or more UV light sources and provide an output accordingly;
at least one flow sensor, wherein the flow sensor is configured to monitor a flow rate related characteristics of an opaque liquid within a fluidic pathway when the UV germicidal treatment system is in use and provide and output accordingly; and
a controller configured for:
receiving a first input relating to the UV output related characteristics of the one or more UV light sources;
receiving a second input relating to the flow related characteristics of the opaque liquid within the fluidic pathway;
determining an UV treatment condition for the UV germicidal treatment system based on the received first and second input and the opaque liquid to be treated; and
controlling the one or more UV light sources and/or the one or more flow rate controllers that control the flow rate based on the determined UV treatment condition.

11. A method for treatment of an opaque liquid treated in an UV germicidal treatment system comprising:
one or more UV light sensors configured to monitor an UV output related characteristics of one or more UV light sources and provide an output accordingly;
at least one flow sensor, wherein the flow sensor is configured to monitor a flow rate related characteristics of an opaque liquid within a fluidic pathway when the UV germicidal treatment system is in use and provide and output accordingly; and
a controller configured for:
receiving a first input relating to the UV output related characteristics of the one or more UV light sources;
receiving a second input relating to the flow related characteristics of the opaque liquid within the fluidic pathway;
determining an UV treatment condition for the UV germicidal treatment system based on the received first and second input and the opaque liquid to be treated; and
controlling the one or more UV light sources and/or the one or more flow rate controllers that control the flow rate based on the determined UV treatment condition;
the method comprising:
optimizing germicidal treatment of the opaque liquid treated in the UV germicidal treatment system.

12. A method for optimizing germicidal treatment of opaque liquids in a UV germicidal treatment system, wherein the method comprises:
providing an opaque liquid through a fluidic pathway in a UV germicidal treatment system;
controlling a flow rate of the opaque liquid through the fluidic pathway via one or more flow rate controllers;
illuminating the fluidic pathway by emitting light in a wavelength range between 180-300 nm from one or more UV light sources;
monitoring and controlling parameters of the UV germicidal treatment system via a surveillance system comprising one or more UV light sensors, at least one flow sensor, and a controller, wherein the UV light sensors monitor an UV output related characteristics of the one or more UV light sources and provide an output accordingly and the at least one flow sensor monitor a flow related characteristics of the opaque liquid within the fluidic pathway and provide an output accordingly, wherein the monitoring and controlling comprises;
providing a first input relating to the UV output related characteristics of the one or more UV light sources to the controller;
providing a second input relating to the flow related characteristics of the opaque liquid within the fluidic pathway to the controller;
determining a UV treatment condition for the UV germicidal treatment system based on the received first and second input and the opaque liquids to be treated; and
controlling the one or more UV light sources and/or the one or more flow rate controllers that control the flow rate based on the determined UV treatment condition.

13. The method according to claim 8, wherein the opaque liquids are opaque liquid food products.

14. The method according to claim 9, wherein the opaque liquids are opaque liquid food products.

15. The method according to claim 14, wherein the microorganisms are selected from the group consisting of bacteria, mold, spores, and viruses.

16. The method according to claim 9, wherein the microorganisms are selected from the group consisting of bacteria, mold, spores, and viruses.
